(19) United States
(12) Patent Application Publication
(10) Pub. No.: US 2017/0281125 A1
(43) Pub. Date: Oct. 5, 2017

(54) PROCESSING SYSTEM, SIGNAL PROCESSING METHOD, AND NON-TRANSITORY STORAGE MEDIUM

(71) Applicant: CANON KABUSHIKI KAISHA, Tokyo (JP)

(72) Inventors: Yukio Furukawa, Sagamihara-shi (JP); Kazuhiko Fukutani, Yokohama-shi (JP); Ryuichi Nanaumi, Tokyo (JP); Yoshiko Nakamura, Kusatsu-shi (JP)

(21) Appl. No.: 15/474,794

(22) Filed: Mar. 30, 2017

(30) Foreign Application Priority Data
Apr. 4, 2016 (JP) 2016-075486

(51) Int. Cl. A61B 8/00; A61B 8/14; A61B 8/08; A61B 5/00

(52) U.S. Cl. CPC A61B 8/4281; A61B 5/0095; A61B 8/14; A61B 8/0825; A61B 8/5269

(57) ABSTRACT

A processing system according to an aspect of the present disclosure includes a transmittance filter obtaining unit and a correcting unit. The transmittance filter obtaining unit obtains a transmittance filter representing complex transmittances corresponding to a plurality of frequencies in the case where an acoustic wave passes through a first medium. The correcting unit corrects an electric signal by using the transmittance filter and obtains a corrected electric signal.

### BACKGROUND OF THE INVENTION

### Field of the Invention

The present disclosure relates to a processing system that processes electric signals derived from acoustic waves.

### Description of the Related Art

Techniques such as photoacoustic tomography (PAT) and ultrasound echo tomography have been proposed as imaging techniques that use reception signals obtained by reception of acoustic waves.

U.S. Pat. No. 6,607,489 discloses an ultrasound echo apparatus that obtains an ultrasound image by taking into account refraction of sound rays due to a compression plate disposed between a subject and a probe for acoustic waves.

In general, acoustic waves propagate through a liquid or a part of a living body, such as a breast, as longitudinal waves. On the other hand, acoustic waves can propagate through a solid not only as longitudinal waves but also as transverse waves. Acoustic waves (longitudinal waves) that have propagated through a living body or a liquid propagate through a solid as a mixture of longitudinal and transverse waves. Further, when the acoustic waves reach a medium, which is a liquid, located closer to a probe after propagating through the solid, the acoustic waves propagate through the liquid medium again as longitudinal waves. Longitudinal and transverse waves have different solid propagation velocities.

Accordingly, an acoustic wave (first acoustic wave) that has propagated through a liquid as a longitudinal wave, a solid as a longitudinal wave, and a liquid as a longitudinal wave and an acoustic wave (second acoustic wave) that has propagated through the liquid as a longitudinal wave, the solid as a transverse wave, and the liquid as a longitudinal wave have different phases and intensities. The first acoustic wave and the second acoustic wave propagate through the medium with mutual interference. The degree of interference is dependent on an angle at which the acoustic wave is incident to the solid. In addition, when the thickness of the solid is substantially equal to or less than the wavelength of the acoustic wave, part of the acoustic wave can pass through the solid (holding member) even if the acoustic wave is incident to the solid at an angle larger than or equal to the critical angle defined by Snell's law. The critical angle is an incident angle for which the transmitted angle is equal to 90 degrees according to Snell's law and is also referred to as an angle of total reflection.

Due to these phenomena, an acoustic wave reaches a probe with its waveform being distorted in accordance with the incident angle of the acoustic wave to a solid.

The method of U.S. Pat. No. 6,607,489 does not take into account this change in the waveform of the acoustic wave that is dependent on the incident angle of the acoustic wave to the compression plate. For this reason, the accuracy of information obtained by using the method of U.S. Pat. No. 6,607,489 decreases because of the distortion of a reception signal due to the change in the waveform of the acoustic wave.

### SUMMARY OF THE INVENTION

Accordingly, an aspect of the present disclosure provides a processing system capable of correcting a distortion of a reception signal based on an acoustic wave due to a waveform distortion that occurs when the acoustic wave passes through a solid.

A processing system according to an aspect of the present disclosure includes a transmittance filter obtaining unit and a correcting unit. The transmittance filter obtaining unit obtains a transmittance filter representing complex transmittances corresponding to a plurality of frequencies in a case where an acoustic wave passes through a first medium. The correcting unit corrects the electric signal by using the transmittance filter and obtains a corrected electric signal.

Further features of the present disclosure will become apparent from the following description of exemplary embodiments with reference to the attached drawings.

### BRIEF DESCRIPTION OF THE DRAWINGS

FIG. 1 is a schematic diagram of a photoacoustic apparatus according to a first exemplary embodiment.

FIG. 2 is a schematic diagram of a configuration of a computer and its peripherals according to the first exemplary embodiment.

FIG. 3 is a flowchart of a subject information obtaining method according to the first exemplary embodiment.

FIGS. 4A to 4C are diagrams for describing a sound ray calculation method according to the first exemplary embodiment.

FIGS. 5A to 5C are diagrams for describing deconvolution processing performed on a reception signal according to the first exemplary embodiment.

FIGS. 6A to 6C are diagrams for describing a propagation time calculation method according to the first exemplary embodiment.

FIGS. 7A to 7D are diagrams illustrating photoacoustic image information according to the first exemplary embodiment.

FIG. 8 is a schematic diagram of a photoacoustic apparatus according to a second exemplary embodiment.

FIG. 9 is a schematic diagram of an ultrasound echo apparatus according to a third exemplary embodiment.

FIGS. 10A and 10B are schematic diagrams illustrating propagation of an ultrasonic wave according to the third exemplary embodiment.

FIG. 11 is a flowchart of a subject information obtaining method according to the third exemplary embodiment.

FIGS. 12A and 12B are schematic diagrams illustrating an incident angle of an ultrasonic wave according to the third exemplary embodiment.

FIG. 13 is a schematic diagram of a photoacoustic apparatus according to a fourth exemplary embodiment.

FIG. 14 is a flowchart illustrating a subject information obtaining method according to the fourth exemplary embodiment.

FIG. 15 illustrates an example of a corrected reception signal according to the fourth exemplary embodiment.

FIG. 16 is a flowchart illustrating another subject information obtaining method according to the fourth exemplary embodiment.

FIG. 17 is a diagram illustrating a position correction amount obtained in accordance with the fourth exemplary embodiment.

FIG. 18 is a diagram illustrating the thickness of a holding cup obtained in accordance with a fifth exemplary embodiment.

FIG. 19 is a diagram illustrating geometric information of the holding cup in the fifth exemplary embodiment.

### DESCRIPTION OF THE EMBODIMENTS

Embodiments of the present disclosure will be described in detail below with reference to the accompanying drawings. The same or substantially the same elements are denoted by the same reference sign, and a description thereof is omitted.

### First Exemplary Embodiment

### Photoacoustic Apparatus

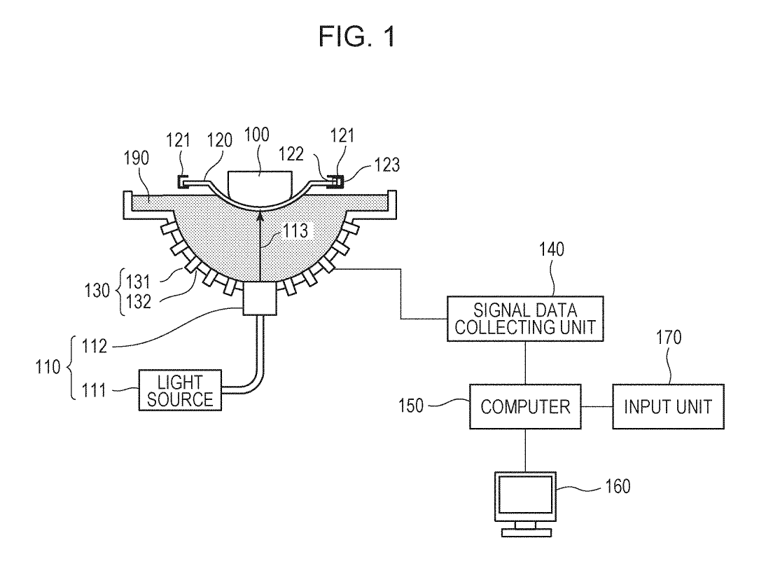

FIG. 1 is a schematic diagram of a photoacoustic apparatus according to a first exemplary embodiment. Components of the photoacoustic apparatus will be described below. The photoacoustic apparatus illustrated in FIG. 1 includes a light irradiation unit 110, a holding cup 120, a probe 130, a signal data collecting unit 140, a computer 150, a display unit 160, and an input unit 170. An object subjected to measurement is a subject 100.

The light irradiation unit 110 irradiates the subject 100 with pulsed light 113, in response to which an acoustic wave is produced in the subject 100. An acoustic wave produced due to light by the photoacoustic effect is also referred to as a photoacoustic wave. The probe 130 receives a photoacoustic wave and outputs electric signals, which are analog signals. The signal data collecting unit 140 converts the electric signals output from the probe 130 as analog signals into digital signals and outputs the digital signals to the computer 150. The computer 150 stores the digital signals output from the signal data collecting unit 140, as signal data derived from the photoacoustic wave.

The computer 150 performs signal processing on the stored digital signals and generates image data representing information (subject information) regarding the subject 100. The computer 150 also performs image processing on the obtained image data and outputs the resultant image data to the display unit 160. The display unit 160 displays an image based on the information regarding the subject 100. A doctor, who is a user, examines the image displayed on the display unit 160 on the basis of the information regarding the subject 100 to make a diagnosis.

The subject information obtained by the photoacoustic apparatus according to the first exemplary embodiment is at least one of produced sound pressure (initial sound pressure) of an acoustic wave, optical energy absorption density, an optical absorption coefficient, and information regarding concentrations of a substance constituting the subject 100, for example. The information regarding concentrations of a substance may be, for example, oxyhemoglobin concentrations, deoxyhemoglobin concentrations, total hemoglobin concentrations, or oxygen saturation. The total hemoglobin concentrations are equal to the sum of the oxyhemoglobin concentrations and deoxyhemoglobin concentrations. The oxygen saturation refers to the fraction of oxyhemoglobin relative to total hemoglobin. The photoacoustic apparatus according to the first exemplary embodiment obtains image data representing values of the above information at respective positions (respective positions in a two-dimensional or three-dimensional space) in the subject 100. That is, the photoacoustic apparatus according to the first exemplary embodiment can be construed as a subject information obtaining apparatus that obtains subject information.

Each component of the photoacoustic apparatus according to the first exemplary embodiment will be described below in detail.

### Light Irradiation Unit 110

The light irradiation unit 110 includes a light source 111 that emits the pulsed light 113 and an optical system 112 that guides the pulsed light 113 emitted from the light source 111 to the subject 100.

The pulsed light 113 emitted by the light source 111 may have a pulse width that is larger than or equal to 1 ns and less than or equal to 100 ns. The pulsed light 113 may have a wavelength in a range from approximately 400 nm to approximately 1600 nm. When a blood vessel near the surface of a living body is imaged at a high resolution, the pulsed light 113 may have a wavelength (longer than or equal to 400 nm and shorter than or equal to 700 nm) that is absorbed at the blood vessel relatively largely. In contrast, when a depth of a living body is imaged, light having a wavelength (longer than or equal to 700 nm and shorter than or equal to 1100 nm) for which an amount of light absorption at background tissues (such as water and fat) of the living body is typically low may be used.

A laser or a light-emitting diode (LED) may be used as the light source 111. In addition, in the case of performing measurement by using light of a plurality of wavelengths, the light source 111 may be capable of tuning the wavelength. Note that in the case of irradiating the subject 100 with light of a plurality of wavelengths, a plurality of light sources that emit light of different wavelengths may be prepared and the subject 100 may be irradiated with the light sequentially from the plurality of light sources. When the plurality of light sources are used, the plurality of light sources are collectively referred to as the light source 111. Various types of laser, such as a solid-state laser, a gas laser, a dye laser, or a semiconductor laser, may be used as the laser. For example, a pulsed laser such as a Nd:YAG laser or an alexandrite laser may be used as the light source 111. In addition, a Ti:sapphire (Ti:sa) laser or an optical parametric oscillator (OPS) laser that uses a laser beam of a Nd:YAG laser as excitation light may be used as the light source 111. In addition, a microwave source may be used as the light source 111.

An optical element, such as a lens, a mirror, or an optical fiber, may be used as the optical system 112. In the case where the subject 100 is a breast or the like, the subject 100 is desirably irradiated with pulsed light having a large beam diameter. Thus, a light-emitting portion of the optical system 112 may include a diffuser plate that diffuses light, for example. On the other hand, in a photoacoustic microscope, the light-emitting portion of the optical system 112 may include a lens or the like to increase the resolution and a beam may be radiated in a focused state. In addition, a fiber bundle, which is a bundle of a plurality of optical fibers, may be used in the optical system 112 that serves as a light-transmitting member. Further, an optical system in which a plurality of hollow waveguides are connected to each other by a mirror-including joint (also referred to as a multi-articular arm) may be used as the optical system 112 that serves as a light-transmitting member.

Note that the light irradiation unit 110 may omit the optical system 112 and may irradiate the subject 100 with the pulsed light 113 directly from the light source 111.

### Holding Cup 120

The holding cup 120, which is a holding member serving as a first medium, is used to hold the shape of the subject 100 during measurement. By holding the subject 100 using the holding cup 120, movement of the subject 100 is successfully suppressed and the position of the subject 100 is successfully kept within the holding cup 120. A resin material, such as polycarbonate, polyethylene, or polyethylene terephthalate, can be used as the material of the holding cup 120.

The holding cup 120 is desirably formed of a material that is hard enough to hold the subject 100. The holding cup 120 may be formed of a material that allows light used in measurement to pass therethrough. The holding cup 120 may be formed of a material having impedance substantially equal to that of the subject 100. If the subject 100 is an object having a curved surface, such as a breast, the holding cup 120 may be formed to have a concave shape. In this case, the subject 100 may be placed in the concave portion of the holding cup 120.

The holding cup 120 is attached to an attachment portion 121. The attachment portion 121 may be configured to allow a plurality of kinds of holding cup 120 to be exchanged in accordance with the size of the subject 100. For example, the attachment portion 121 may be configured to allow holding cups having different radii of curvature, different curvature centers, etc. to be exchanged.

In addition, a tag 122 in which the specifications of the holding cup 120 are registered may be attached to the holding cup 120. For example, specifications such as the radius of curvature, the curvature center, the longitudinal-wave sound velocity, the transverse-wave sound velocity, and identification (ID) of the holding cup 120 may be registered in the tag 122. The specifications registered in the tag 122 are read by a reader unit 123 and are transferred to the computer 150. The reader unit 123 may be disposed at the attachment portion 121 to allow the tag 122 to be read easily when the holding cup 120 is attached to the attachment portion 121. For example, the tag 122 may be a barcode, and the reader unit 123 may be a barcode reader.

### Probe 130

The probe 130 serving as a receiving unit includes transducers 131 each of which receives an acoustic wave and outputs an electric signal and a support 132 that supports the transducers 131.

A piezoelectric ceramic material (typically, lead zirconate titanate (PZT)), a polymer piezoelectric film material (typically, polyvinylidene difluoride (PVDF)), or the like can be used as a material of the transducers 131. In addition, elements other than piezoelectric elements may be used. For example, capacitive transducers (typically, capacitive micro-machined ultrasonic transducers (CMUTs)), transducers using a Fabry-Pérot interferometer, or the like can be used. Note that any type of transducers may be used as long as the transducers are capable of receiving an acoustic wave and outputting an electric signal. In addition, signals obtained by the transducers 131 are time-resolved signals. That is, the amplitude of a signal obtained by each of the transducers 131 serving as receiving elements represents a value based on sound pressure (e.g., a value proportional to the sound pressure) received by the transducer 131 at each time point.

Since frequency components of a photoacoustic wave are typically in a range from 100 KHz to 100 MHz, transducers capable of detecting these frequencies may be used as the transducers 131.

The support 132 may be composed of a material having high mechanical strength, such as a metal material or a plastic material. In the first exemplary embodiment, the support 132 has a shape of a hemispherical shell so as to be able to support the plurality of transducers 131 on the hemispherical shell. In this case, the axes of directivity of the transducers 131 disposed on the support 132 concentrate at around the curvature center of the hemisphere. When an image is obtained by using electric signals output from the plurality of transducers 131, the image quality at around the curvature center is high. Note that the support 132 may be configured in any manner as long as the support 132 is capable of supporting the transducers 131. The support 132 may be a flat or curved surface on which the plurality of transducers 131 are disposed in a manner of so-called 1D array, 1.5 array, 1.75 array, or 2D array.

The support 132 may also function as a container that stores an acoustic matching material 190. That is, the support 132 may be used as a container for placing the acoustic matching material 190 between the transducers 131 and the subject 100.

The probe 130 may also include an amplifier that amplifies time-series analog signals output from the respective transducers 131. In addition, the probe 130 may include an analog/digital (A/D) converter that converts time-series analog signals output from the respective transducers 131 into time-series digital signals. That is, the probe 130 may include the signal data collecting unit 140 (described later).

Ideally, the transducers 131 are desirably disposed to entirely surround the subject 100 so as to be able to detect an acoustic wave at various angles. However, if it is difficult to dispose the transducers 131 to entirely surround the subject 100 because the subject 100 is large, the arrangement state may be made closer to the entirely surrounding state by disposing the transducers 131 on the hemispherical support 132 as illustrated in FIG. 1. Note that the arrangement state and the number of transducers 131 and the shape of the support 132 may be optimized in accordance with the subject 100, and any type of probe 130 may be used in embodiments of the present disclosure.

### Signal Data Collecting Unit 140

The signal data collecting unit 140 includes an amplifier that amplifies electric signals that are analog signals output from the respective transducers 131 and an A/D converter that converts the analog signals output from the amplifier into digital signals. The signal data collecting unit 140 may be implemented by a field programmable gate array (FPGA) chip or the like. The digital signals output from the signal data collecting unit 140 are stored in a storage unit of the computer 150. The signal data collecting unit 140 is also referred to as a data acquisition system (DAS). The term “electric signals” used herein include analog signals and digital signals. The signal data collecting unit 140 may be connected to a light detection sensor attached to the light-emitting portion of the light irradiation unit 110 and may start processing in synchronization in response to emission of the pulsed light 113 from the light irradiation unit 110.

### Computer 150

The computer 150 includes a processing unit, a storage unit, and a control unit. Functions of the respective components will be described when the flow of processing is described.

The storage unit may be implemented by non-transitory storage medium such as a read-only memory (ROM), a magnetic disk, or a flash memory. In addition, the storage unit may be a volatile memory such as a random access memory (RAM). Note that a storage medium that stores a program is a non-transitory storage medium.

Units that serve as the processing unit and carry out an arithmetic function can be implemented by a processor such as a central processing unit (CPU), a graphics processing unit (GPU), or a digital signal processor (DSP); or an arithmetic circuit such as an FPGA chip. These units may be implemented not only by a single processor or arithmetic circuit but also by a plurality of processors or arithmetic circuits.

The control unit is implemented by an arithmetic element such as a CPU. The control unit controls operations of the components of the photoacoustic apparatus. The control unit may receive instruction signals based on various operations, such as the start of measurement, from the input unit 170 and control the components of the photoacoustic apparatus. In addition, the control unit reads program code stored in the storage unit and controls operations of the components of the photoacoustic apparatus.

The computer 150 may be a workstation designed for this purpose. In addition, the components of the computer 150 may be implemented by different hardware components. In addition, at least some of the components of the computer 150 may be implemented by a single hardware component.

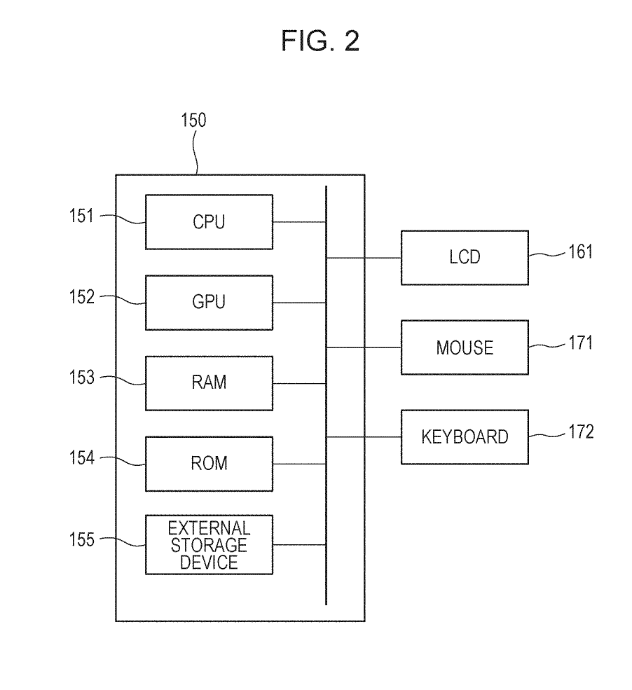

FIG. 2 illustrates a specific configuration of the computer 150 according to the first exemplary embodiment. The computer 150 according to the first exemplary embodiment includes a CPU 151, a GPU 152, a RAM 153, a ROM 154, and an external storage device 155. In addition, a liquid crystal display (LCD) 161 that serves as the display unit 160 and a mouse 171 and a keyboard 172 that serve as the input unit 170 are connected to the computer 150.

The computer 150 and the plurality of transducers 131 may be provided after being contained in a single housing. Part of signal processing may be performed by the computer contained in the housing, and the rest of signal processing may be performed by a computer provided outside the housing. In this case, the computer contained in the housing and the computer externally provided can be collectively referred to as the computer 150 according to the first exemplary embodiment.

A system that processes signals output from the transducers 131 is collectively referred to as a processing system. The processing system may include a plurality of processing apparatuses.

### Display Unit 160

The display unit 160 is a display such as an LCD or an organic electroluminescence display. The display unit 160 is an apparatus that displays an image based on subject information or the like obtained by the computer 150, a value at a specific position, or the like. The display unit 160 may display a graphical user interface (GUI) used to operate an image or the apparatus. The subject information may be displayed after image processing (such as adjustment of luminance values) is performed on the subject information by the display unit 160 or the computer 150.

### Input Unit 170

The input unit 170 can be implemented by devices that can be operated by the user, such as a mouse and a keyboard. In addition, the display unit 160 may include a touch panel, and the display unit 160 may be used as the input unit 170.

Note that the components of the photoacoustic apparatus may be implemented as separate apparatuses or a single integrated apparatus. In addition, at least some of the components of the photoacoustic apparatus may be implemented as a single apparatus.

### Acoustic Matching Material 190

The acoustic matching material 190, which is a second medium, is not a component of the photoacoustic apparatus; however, the acoustic matching material 190 will be described. The acoustic matching material 190 is a material that allows an acoustic wave to propagate through a space between the subject 100 and the transducers 131. The acoustic matching material 190 may be implemented by a deformable material and may deform upon contact of the subject 100 onto the acoustic matching material 190. That is, the acoustic matching material 190 may be implemented by a material that is deformable in accordance with the subject 100 in order to minimize a gap between the subject 100 and the transducers 131. The acoustic matching material 190 may be a material for which attenuation of an acoustic wave is small. A material having acoustic impedance that is between the acoustic impedance of the subject 100 and the acoustic impedance of the transducers 131 may be used as the acoustic matching material 190. In particular, a material having acoustic impedance closer to that of the subject 100 may be selected. If irradiation light passes through the acoustic matching material 190, the acoustic matching material 190 may be transparent for the irradiation light. Water, ultrasound gel, or the like may be used as the acoustic matching material 190.

In the first exemplary embodiment, the support 132 functions as a container that stores the acoustic matching material 190. Note that the photoacoustic apparatus may include a container capable of storing the acoustic matching material 190 between the transducers 131 and the subject 100 other than the support 132. A plastic container, a metal container, or the like may be used as the container.

### Subject 100

The subject 100 is not a component of the photoacoustic apparatus; however, the subject 100 will be described below. The photoacoustic apparatus according to the first exemplary embodiment may be used for the purpose of diagnosis of a malignant tumor or a blood vessel disease of persons and animals and follow-up of the chemotherapy. Accordingly, the subject 100 is expectedly a living body, more specifically, a diagnosis-target site, such as a breast, cervix, or abdomen of persons or animals. For example, if a person is subjected to measurement, an optical absorber may be a blood vessel containing lots of oxyhemoglobin or deoxyhemoglobin or a new blood vessel created near a tumor. In addition, an optical absorber may be a plaque on the carotid artery wall or the like. In addition, an optical absorber may be a dye such as methylene blue (MB) or indocyanine green (ICG), fine gold particles, or an externally introduced substance that is accumulation of these substances or that chemically modifying these substances.

The first exemplary embodiment will be described by using the photoacoustic apparatus illustrated in FIG. 1. The light source 111 is a Ti:sapphire laser that produces the pulsed light 113 having a wavelength of 800 nm, a pulse width of 20 ns, a repetition frequency of 10 Hz, and a pulse energy of 150 mJ. The optical system 112 includes a fiber bundle that serves as a light-transmitting member and a concave lens that serves as a light irradiation unit and increases the beam diameter of light. An end of the fiber bundle is connected to a concave lens.

The support 132 supports the concave lens serving as the light irradiation unit and the transducers 131. The inner surface of the support 132 that supports the transducers 131 has a hemispherical shape of a radius of 127 mm, and 512 transducers 131 are disposed along the hemispherical surface. Water serving as the acoustic matching material 190 is stored in the support 132.

The holding cup 120 is composed of a polyethylene terephthalate resin having a thickness of 0.5 mm. For example, the subject 100 may be a breast of a person or the like. The portion of the holding cup 120 to be in contact with the subject 100 is a hemispherical surface having a radius of 230 mm. A space between the subject 100 and the holding cup 120 is filled with ultrasound gel (not illustrated) that serves as an acoustic matching material for acoustic matching.

In the first exemplary embodiment, 512 transducers 131 each including a piezoelectric element having an element size of 3 mm per side and a center detection frequency of 2 MHz are disposed on the hemispherical surface.

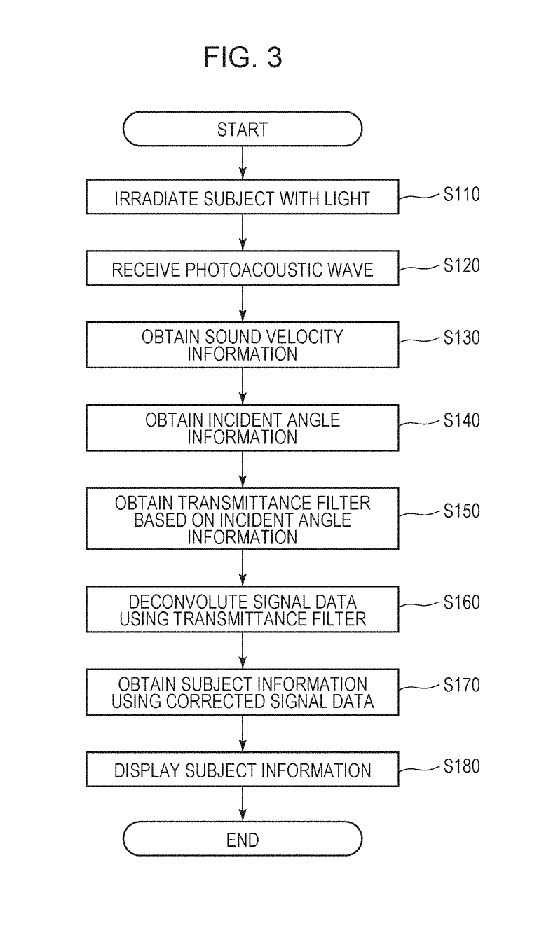

A subject information obtaining method according to the first exemplary embodiment will be described below with reference to a flowchart of FIG. 3.

S110: Irradiating Subject with Light

The subject 100 is irradiated with, via the optical system 112, the pulsed light 113 that is light produced by the light source 111. The pulsed light 113 is absorbed inside the subject 100, and a photoacoustic wave is produced due to the photoacoustic effect.

### S120: Receiving Photoacoustic Wave

In step S120, the probe 130 receives the photoacoustic wave and outputs electric signals from the respective transducers 131. The output electric signals (reception signals) are transferred to the computer 150. In the first exemplary embodiment, the signal data collecting unit 140 performs A/D conversion at a sampling rate of 20 MHz to convert the individual analog electric signals output from the respective transducers 131 into digital signals and obtains signal data. In addition, the signal data collecting unit 140 sets a signal corresponding to a timing at which the subject 100 is irradiated with the pulsed light 113 as the 0-th sample and stores the obtained signal data by excluding signals of the 0-th to 999-th samples in order to exclude redundant information. That is, the signal data collecting unit 140 obtains signal data of 2048 samples from the 1000-th sample to 3047-th sample.

Note that the photoacoustic apparatus may include a position determining unit that determines the position at which the probe 130 is located at the time of reception of a photoacoustic wave. For example, a magnetic sensor included in the probe 130 or an encoder of a stage that mechanically moves the probe 130 may be used as the position determining unit. For example, in response to emission of light from the light irradiation unit 110, the position determining unit may output position information regarding the position of the probe 130 at that time to the computer 150. The computer 150 may use the position information regarding the position of the probe 130 thus obtained in processing of S140 to S170 (described below).

### S130: Obtaining Sound Velocity Information

The computer 150 serving as a sound velocity obtaining unit obtains a longitudinal-wave sound velocity c₁ in the subject 100, a longitudinal-wave sound velocity c_{2L} in the holding cup 120 (in the first medium), a transverse-wave sound velocity c_{2T} in the holding cup 120, and a longitudinal-wave sound velocity c₃ in the acoustic matching material 190 (in the second medium).

As for the longitudinal-wave sound velocity c_{2L} and the transverse-wave sound velocity c_{2T} in the holding cup 120, data obtained by measurement performed in advance may be stored in the storage unit. The computer 150 may obtain these sound velocities in step S130 by reading the stored data from the storage unit. A relationship formula or relationship table of the longitudinal-wave sound velocity c_{2L} and the transverse-wave sound velocity c_{2T} in the holding cup 120 relative to temperature of the holding cup 120 may be stored in advance in the storage unit. In step S130, a temperature measuring unit may measure the temperature of the holding cup 120, and the computer 150 may obtain the sound velocities associated with the measured temperature in accordance with the relationship formula or the relationship table.

In addition, the user may input the sound velocities in the holding cup 120 by using the input unit 170, and the computer 150 may obtain the sound velocities by receiving the input information.

In addition, the computer 150 may obtain the sound velocities in the holding cup 120 attached to the attachment portion 121. For example, the reader unit 123 reads information regarding the sound velocities in the holding cup 120 registered in the tag 122 attached to the holding cup 120 and may transfer the information to the computer 150. The computer 150 may obtain information regarding the sound velocities in the holding cup 120, which has been read by the reader unit 123. In addition, the user may input the ID assigned to the holding cup 120 by using the input unit 170, and the computer 150 may obtain the sound velocities in the holding cup 120 by reading from the storage unit the sound velocities associated with the input ID.

The longitudinal-wave sound velocity in the subject 100 may be obtained by using a method similar to that used for the longitudinal-wave sound velocity and the transverse-wave sound velocity in the holding cup 120. However, since the longitudinal-wave sound velocity in the subject 100 varies from subject 100 to subject 100, new data is desirably obtained for each subject 100. The computer 150 may obtain the sound velocity in the subject 100 by using a signal derived from an acoustic wave produced in the subject 100. For example, the computer 150 sets a dummy sound velocity, generates a reconstructed image from the signal by using the dummy sound velocity, and evaluates the image quality (such as contrast or resolution) of the reconstructed image. The computer 150 repeatedly performs this processing by using different dummy sound velocities and sets, as the sound velocity in the subject 100, the dummy sound velocity for which the image quality of the reconstructed image is higher than a predetermined threshold. Other than this method, the computer 150 may obtain the sound velocity in the subject 100 by evaluating, using a dummy sound velocity, a variance of the signals output from the plurality of transducers 131 which are derived from an acoustic wave produced at a specific position. In this case, the computer 150 obtains, as the sound velocity in the subject 100, the dummy sound velocity for which the variance of the signals is smaller than a predetermined threshold. Any method allowing the sound velocity in the subject 100 to be obtained by using the signals derived from an acoustic wave produced in the subject 100 to be obtained may be used other than the above method. In addition, known values stored in the storage unit may be used as sound velocities in the components other than the subject 100. In addition, when the holding cup 120 is sufficiently thin (thickness is less than the wavelength of the acoustic wave), the computer 150 may obtain the sound velocity in the subject 100 by ignoring the presence of the holding cup 120 and assuming that the sound velocity in the acoustic matching material 190 is known. According to this method, the sound velocity that is specific to each subject 100 can be obtained without increasing the scale of the apparatus. The computer 150 may obtain the sound velocity in the subject 100 by using any other known methods.

The longitudinal-wave sound velocity in the acoustic matching material 190 may be obtained by using a method similar to that used for the longitudinal-wave sound velocity and the transverse-wave sound velocity in the holding cup 120. The computer 150 may obtain the sound velocity in the acoustic matching material 190 by using a signal derived from an acoustic wave produced in the subject 100. In addition, the temperature measuring unit may measure the temperature of the acoustic matching material 190, and the computer 150 may obtain the sound velocity corresponding to the measured temperature in accordance with a relationship formula or relationship table. The computer 150 may obtain the sound velocity in the acoustic matching material 190 by using any other known methods.

The above description is given of the example in which the computer 150 obtains the sound velocities; however, the computer 150 may obtain any parameters from which the sound velocities can be estimated in this step. For example, since the sound velocity can be determined from a density ρ and a bulk modulus K, the computer 150 may obtain the density ρ and the bulk modulus K and estimate the sound velocity from these parameters in this step. Herein, the term “sound velocity information” not only refers to propagation speed (sound velocity) of a longitudinal or transverse wave but also refers to parameters from which the sound velocity can be estimated.

### S140: Obtaining Incident Angle Information

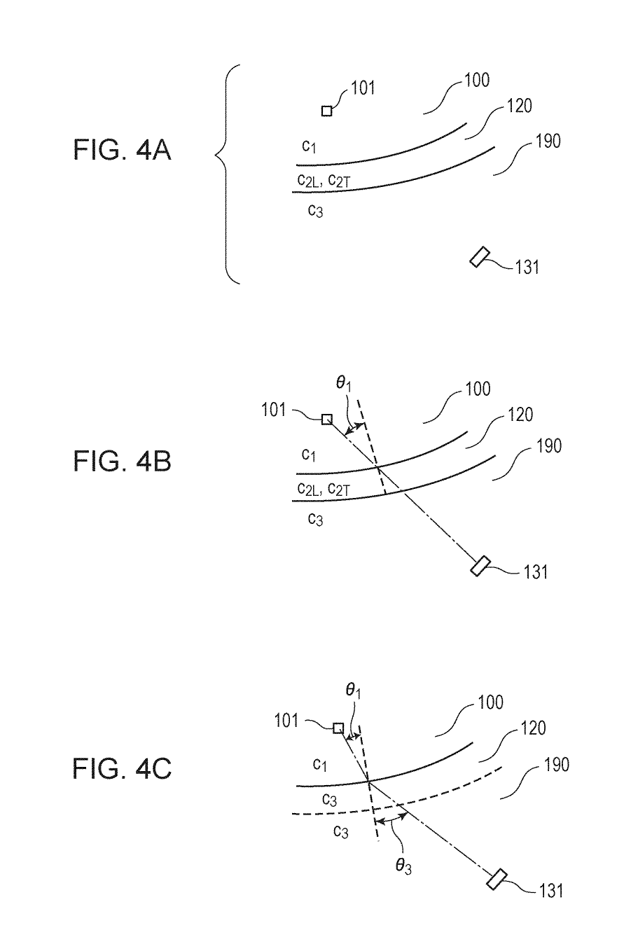

The computer 150 serving as an angle obtaining unit obtains incident angle information regarding an incident angle at which the acoustic wave that is to reach the transducer 131 is incident to the holding cup 120 in the case where the holding cup 120 and the transducer 131 are arranged as illustrated in FIG. 4A. In the first exemplary embodiment, the description will be given of an example where the computer 150 obtains the incident angle information regarding an incident angle at which an acoustic wave produced at a position corresponding to a voxel 101 to be imaged later is incident to the holding cup 120.

In this step, the computer 150 calculates a sound ray of an acoustic wave by assuming that the holding cup 120 is absent. Then, the computer 150 obtains, as the incident angle information, an incident angle at which the calculated sound ray is incident to the holding cup 120.

The description will be given of an example where the sound ray of the acoustic wave produced at the position corresponding to the voxel 101 is linearly approximated as illustrated in FIG. 4B. When a difference between the sound velocity in the subject 100 and the sound velocity in the acoustic matching material 190 is small or when the size of the voxel 101 is relatively large, a sound ray may be linearly approximated as illustrated in FIG. 4B. The computer 150 may employ linear approximation when a distance (difference in traveling time×sound velocity) corresponding to a difference between traveling time in the case where refraction is taken into account and traveling time in the case where linear approximation is performed is sufficiently smaller than the size of the voxel 101. For example, when the distance value is smaller than 1/10 the size of the voxel 101, the distance corresponding to the difference in traveling time is sufficiently small.

For example, suppose that the sound velocity in the subject 100 is 1460 m/s, the sound velocity in the acoustic matching material 190 is 1480 m/s, and the size of the voxel 101 is 0.25 mm. In this case, for an incident angle of 50 degrees, a difference in traveling time between the case where refraction is taken into account and the case where linear approximation is performed is 2.8 ns, which is converted into a distance of approximately 4.2 μm. Since this value is smaller than 1/10 the size of the voxel 101 (25 μm), linear approximation may be performed in this case. Since the difference in traveling time is proportional to a square of a difference in sound velocity and is proportional to a square of a value of tan(incident angle), the computer 150 may choose whether to take refraction into account or to perform linear approximation in consideration of this fact.

The computer 150 connects the voxel 101 and the transducer 131 by using a straight line and obtains, as the incident angle information, an angle θ₁ between this straight line and the holding cup 120 as illustrated in FIG. 4B. At that time, the computer 150 serving as a position obtaining unit may obtain the position information of the transducer 131 and the shape information of the holding cup 120 by reading these pieces of information stored in the storage unit in advance. In addition, the computer 150 may use the shape information obtained by using a shape obtaining unit that obtains the shape information of the holding cup 120.

On the other hand, when the difference between the sound velocity in the subject 100 and the sound velocity in the acoustic matching material 190 is large or when the size of the voxel 101 is relatively small, the computer 150 calculates a sound ray by taking refraction into account as illustrated in FIG. 4C. The computer 150 may employ calculation in which refraction is taken into account when a distance (difference in traveling time×sound velocity) corresponding to a difference in traveling time between the case where refraction is taken into account and the case where linear approximation is performed is sufficiently larger than the size of the voxel 101. For example, when the distance value is larger than 1/10 the size of the voxel 101, the distance corresponding to the difference in traveling time is sufficiently larger than the size of the voxel 101.

For example, suppose that the sound velocity in the subject 100 is 1400 m/s, the sound velocity in the acoustic matching material 190 is 1480 m/s, and the size of the voxel 101 is 0.25 mm. In this case, for the incident angle of 50 degrees, the difference in traveling time between the case where refraction is taken into account and the case where linear approximation is performed is 48 ns, which is converted into a distance of approximately 70 μm. Since this value is larger than 1/10 the size of the voxel 101 (25 μm), refraction may be taken into account in this example.

The computer 150 calculates a sound ray that connects the voxel 101 and the transducer 131 in accordance with Snell's law represented by Equation (1) and obtains, as the incident angle information, an angle θ₁ between the sound ray and the holding cup 120.

$$\frac{sin ( {θ}_{1} )}{{c}_{1}} = \frac{sin ( {θ}_{3} )}{{c}_{3}} \qquad ( 1 )$$

In Equation (1), θ₃ represents an angle between the sound ray in the acoustic matching material 190 and the holding cup 120 and is also referred to as a propagation angle below.

The computer 150 performs the processing of this step for all the voxels 101 and all the transducers 131 subjected to the processing.

Note that the computer 150 serving as the position obtaining unit may obtain a positional relationship between a certain voxel and a certain transducer and may associate the incident angle information determined by this positional relationship with the proximal voxel or transducer. In addition, the computer 150 may perform interpolation processing on the incident angle information determined by the positional relationship between the certain voxel and the certain transducer and may obtain the incident angle information corresponding to a combination of another voxel and the transducer. In these cases, the computer 150 need not necessarily calculate sound rays for all the combinations of a voxel and a transducer.

### S150: Obtaining Transmittance Filter Based on Incident Angle Information

The computer 150 serving as a transmittance filter obtaining unit obtains a transmittance filter for a reception signal on the basis of the incident angle information obtained in S140. In the first exemplary embodiment, the transmittance filter represents transmittances in the case where an acoustic wave passes through the holding cup 120 from the side closer to the subject 100 to the side closer to the transducers 131. Since the transmittance is dependent on the frequency of the acoustic wave, the transmittance is obtained for each frequency. In addition, since the transmittance contains phase information, it is represented using a complex number. Hereinafter, complex transmittances corresponding to a plurality of frequencies are collectively referred to as a transmittance filter. Note that the frequency band of the transmittance filter may be determined in accordance with frequency components included in the acoustic wave handled in embodiments of the present disclosure.

When an acoustic wave passes through the holding cup 120, the transmitted wave distorts because of multiple reflections caused by a first surface (surface closer to the subject 100) and a second surface (surface closer to the probe 130) of the holding cup 120. The inventor has experimentally found that the influence of multiple reflections is small if the holding cup 120 has a curved surface as in the first exemplary embodiment. In this case, the transmittance filter may be calculated without taking into account multiple reflections.

The computer 150 may choose whether to apply a transmittance filter for which multiple reflections are taken into account or to apply a transmittance filter for which multiple reflections are not taken into account on the basis of the radius of curvature of the holding cup 120. For example, if the holding cup 120 has a radius of curvature equal to or smaller than 300 mm, the computer 150 may apply the transmittance filter for which multiple reflections are not taken into account. On the other hand, the computer 150 may apply the transmittance filter for which multiple reflections are taken into account if the holding cup 120 has a radius of curvature larger than 300 mm. In addition, the computer 150 may apply the transmittance filter for which multiple reflections are not taken into account if the holding cup 120 has a radius of curvature equal to or smaller than 100 mm. In addition, the computer 150 may apply the transmittance filter for which multiple reflections are taken into account if the holding cup 120 has a radius of curvature larger than 100 mm.

In addition, the computer 150 may choose whether to apply a transmittance filter for which multiple reflections are taken into account or to apply a transmittance filter for which multiple reflections are not taken into account on the basis of the incident angle information. For example, a transmittance filter for which multiple reflections are taken into account may be applied if the incident angle is in a range around the critical angle (e.g., ±5° from the critical angle); otherwise, a transmittance filter for which multiple reflections are not taken into account may be applied.

In addition, the computer 150 may choose whether to apply a transmittance filter for which multiple reflections are taken into account or to apply a transmittance filter for which multiple reflections are not taken into account on the basis of the thickness of the holding cup 120.

That is, the computer 150 may be configured to select one of transmittance filters different from each other (i.e., a transmittance filter for which multiple reflections are taken into account and a transmittance filter for which multiple reflections are not taken into account).

How a transmittance filter for which multiple reflections are not taken into account is calculated will be described in the first exemplary embodiment. How a transmittance filter for which multiple reflections are taken into account is calculated will be described in a second exemplary embodiment.

Let c₁ denote the (longitudinal-wave) sound velocity in the subject 100, c_{2L} and c_{2T} respectively denote the longitudinal-wave and transverse-wave sound velocities in the holding cup 120, c₃ denote the (longitudinal-wave) sound velocity in the acoustic matching material 190, θ₁ denote the incident angle, and T denote the thickness of the holding cup 120. In addition, let Z₁ denote the acoustic impedance of the subject 100, Z_{2L} and Z_{2T} respectively denote the acoustic impedances of the holding cup 120 for a longitudinal wave and a transverse wave, and Z₃ denote the acoustic impedance of the acoustic matching material 190. The incident angle θ₁ is a value calculated in S140.

The propagation angles θ_{2L} and θ_{2T} of a longitudinal wave and a transverse wave in the holding cup 120 and the propagation angle θ₃ in the acoustic matching material 190 have a relationship represented by Equation (2) according to Snell's law.

$$\frac{sin {θ}_{1}}{{c}_{1}} = \frac{sin {θ}_{2 L}}{{c}_{2 L}} = \frac{sin {θ}_{2 T}}{{c}_{2 T}} = \frac{sin {θ}_{3}}{{c}_{3}} \qquad ( 2 )$$

For example, if c_{2L} is larger than c₁, sin θ_{2L} may be larger than 1 (sin θ_{2L}>1) in some cases. At that time, cos θ_{2L} represents an imaginary number. The sign is set so that attenuation occurs in response to propagation in a direction perpendicular to the surface of the holding cup 120. That is, Equation (3) is set.

cos θ_{2L}=−i\sqrt{sin²θ_{2L}−1} (3)

Likewise, when cos θ_{2T} represents an imaginary number, Equation (4) is set.

cos θ_{2T}=−i\sqrt{sin²θ_{2T}−1} (4)

Let t_{12L} denote a complex amplitude transmittance for an acoustic wave (longitudinal wave) that is incident to the first surface of the holding cup 120 at the incident angle θ₁ from the side closer to the subject 100 and passes through the first surface as a longitudinal wave toward the holding cup 120. In addition, let t_{12T} denote a complex amplitude transmittance for the acoustic wave that passes through the first surface as a transverse wave. In this case, the complex amplitude transmittances t_{12L} and t_{12T} can be respectively determined by using Equations (5) and (6).

$$\begin{matrix}{t}_{12 L} = \frac{2 \frac{{Z}_{1}}{cos {θ}_{2 L}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 5 ) \\ {t}_{12 T} = \frac{- 2 \frac{{Z}_{1}}{cos {θ}_{2 L}} sin 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 6 )\end{matrix}$$

As the acoustic wave propagates through the holding cup 120 from the first surface to the second surface, the phase of the acoustic wave shifts. The phase shift is dependent on the frequency of the acoustic wave. If cos θ_{2L} or cos θ_{2T} represents an imaginary number, attenuation occurs. Let ω denote the angular frequency of the acoustic wave and _{2L} and φ_{2T} respectively denote phase shift amounts (attenuation amounts) for the longitudinal wave and the transverse wave. Then, the phase shift amounts φ_{2L} and φ_{2T} can be respectively determined by using Equations (7) and (8).

$$\begin{matrix}{φ}_{2 L} ( ω ) = exp ( - i \frac{ω T cos {θ}_{2 L}}{{c}_{2 L}} ) & ( 7 ) \\ {φ}_{2 T} ( ω ) = exp ( - i \frac{ω T cos {θ}_{2 T}}{{c}_{2 T}} ) & ( 8 )\end{matrix}$$

Let t_{23L} and t_{23T} respectively denote complex amplitude transmittances for the longitudinal and transverse acoustic waves that are incident on the second surface of the holding cup 120 and pass through the second surface as a longitudinal wave toward the acoustic matching material 190. In this case, the complex amplitude transmittances t_{Z3L} and t_{23T} are respectively determined by using Equations (9) and (10).

$$\begin{matrix}{t}_{23 L} = \frac{2 \frac{{Z}_{2 L}}{cos {θ}_{3}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 9 ) \\ {t}_{23 T} = \frac{- 2 \frac{{Z}_{2 T}}{cos {θ}_{3}} sin 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 10 )\end{matrix}$$

The transmittance filter f(ω) for the holding cup 120 relative to the angular frequency ω can be represented by Equation (11).

$$f ( ω ) ( {t}_{12 L} {φ}_{2 L} ( ω ) {t}_{23 L} + {t}_{12 T} {φ}_{2 T} ( ω ) {t}_{23 T} ) exp ( i \frac{ω T cos {θ}_{3}}{{c}_{3}} ) \qquad ( 11 )$$

In Equation (11), the “exp” term at the latter part is provided to correct the amount of phase shift that occurs when the acoustic matching material 190 is present in a region of the holding cup 120. With the presence of this “exp” term, a reception signal that is equivalent to a signal obtained in the case where the region of the holding cup 120 is replaced with the acoustic matching material 190 is obtained when correction of the reception signal to be described in S160 is performed.

As described above, the computer 150 successfully obtains the transmittance filter by using the incident angle information obtained in S140. The computer 150 according to the first exemplary embodiment is also able to obtain the transmittance filter by further using the thickness of the holding cup 120, the longitudinal-wave and transverse-wave sound velocities in the holding cup 120, the longitudinal-wave sound velocity in the subject 100, and the longitudinal-wave sound velocity in the acoustic matching material 190.

During deconvolution performed in S160 (described later), processing is performed on a frequency-domain reception signal obtained by performing discrete Fourier transform on the reception signal. In the first exemplary embodiment, discrete Fourier transform of the reception signal yields 2048 pieces of frequency information from −9990234.375 Hz to 10000000 Hz at an interval of 9765.625 Hz. Accordingly, in this step, the transmittance filter represented by Equation (11) is calculated for 2048 angular frequencies needed in the processing in S160 (described later).

While the example where the computer 150 calculates the transmittance filter for all the propagation paths of the acoustic wave each time has been described above, the method for obtaining the transmittance filter is not limited to this one. For example, a relationship formula or relationship table representing relationships between an incident angle and a transmittance filter may be stored in the storage unit of the computer 150. Then, the computer 150 may determine the transmittance filter corresponding to the incident angle by referring to the relationship formula or relationship table stored in the storage unit on the basis of the incident angle information obtained in S140. The transmittance filter corresponding to the incident angle not included in the relationship table may be generated by interpolation based on the preceding data and the following data. With such a configuration, the calculation time for obtaining the transmittance filter is successfully reduced.

This step is performed for all the transducers 131. The processing of this step may be performed sequentially for the respective transducers 131 in terms of time, or the processing may be performed in parallel for the plurality of transducers 131 simultaneously.

### S160: Performing Deconvolution on Signal Data by Using Transmittance Filter

The computer 150 serving as a correcting unit performs deconvolution on the signal data obtained in S120, by using the transmittance filter obtained in S150 as a response function and obtains corrected signal data. This processing is, in other words, processing of obtaining corrected signal data by superimposing a deconvolution filter derived from the transmittance filter onto the signal data. The deconvolution-filter superimposition processing effectively corrects a change in the acoustic wave that occurs when the acoustic wave passes through the holding cup 120. That is, as a result of this deconvolution processing, signal data equivalent to signal data obtained in the case where the transducer 131 receives the acoustic wave without via the holding cup 120 can be obtained. The correcting unit successfully removes or reduces the distortion of the signal data that occurs when the acoustic wave passes through the holding cup 120.

Further, the computer 150 may have a function of obtaining reception characteristics (impulse responses) of the transducers 131 in advance and of performing deconvolution by using the reception characteristics as a response function. With this processing, degradation of signal data due to impulse responses is successfully corrected. Either deconvolution using the impulse responses as the response function or deconvolution using the transmittance filter as the response function may be performed first or may be performed simultaneously.

This calculation performed by the computer 150 will be described below.

Let S₀(t) denote a reception signal of a certain transducer 131, F[S₀] (ω) denote a Fourier transform signal of the reception signal S₀(t), and f(ω) denote the transmittance filter. In addition, let F⁻⁻¹[ ] denote inverse Fourier transform.

In the ideal state in which there are no noise and no deviation from the designed state, a corrected reception signal S(t) can be represented by

$$S ( t ) = real ( {F}^{- 1} [ \frac{F [ {S}_{0} ] ( ω )}{f ( ω )} ] ) , \qquad ( 12 )$$

where real( ) is a function for extracting the real part alone.

Equation (12) is not practical because there is noise or division by zero is performed for the incident angle for which f(ω) is calculated to be zero in a real situation. Accordingly, a filter generally known as the Wiener filter is used as the deconvolution filter.

Let D(ω) denote the deconvolution filter. Then, the deconvolution filter D(ω) is determined in a manner represented by Equation (13).

$$D ( ω ) = \frac{conj ( f ( ω ) )}{conj ( f ( ω ) ) \cdot f ( ω ) + C} \qquad ( 13 )$$

In Equation (13), conj denotes a complex conjugate and C denotes a constant. The constant C is set empirically so as not to degrade the signal considerably. The constant C may be generally set to a several percent of the maximum value of |f(ω)|.

The corrected reception signal S(t) is determined by performing calculation represented by Equation (14) using the deconvolution filter D(ω).

S(t)=real(F⁻¹[F[S₀](ω)·D(ω)]) (14)

The corrected reception signal S(t) thus obtained is substantially equivalent to a reception signal obtained when the region of the holding cup 120 is replaced with the acoustic matching material 190.

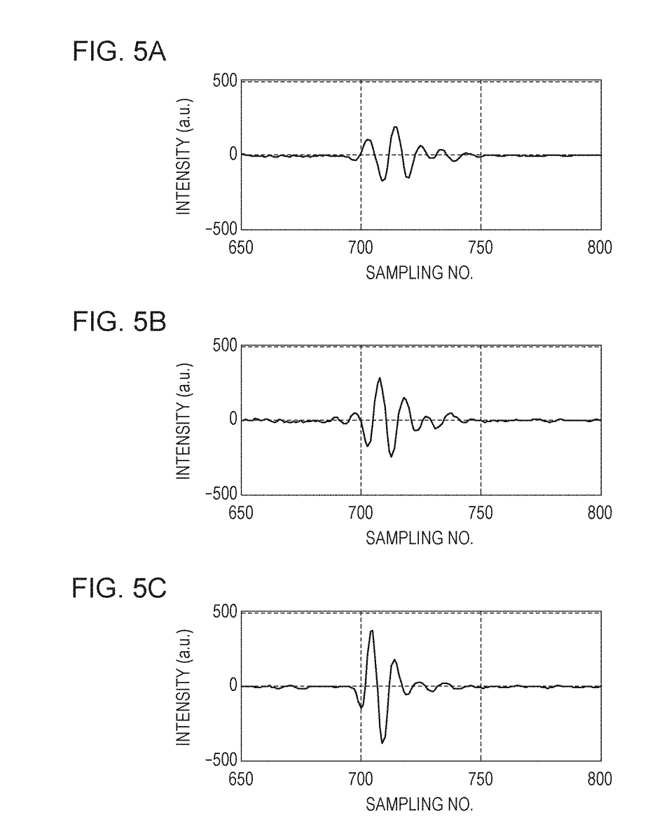

FIGS. 5A to 5C illustrate an example in which signal correction is performed by using a reception signal actually measured. In this example, a rubber ball having a diameter of 0.3 mm, which serves as a sound source (optical absorber), is placed adjacent to the center of the spherical surface on which the transducers 131 are disposed. The space around the sound source is filled with water. As for the sound velocities, c₁=1486 m/s, c₃=1486 m/s, c_{2L}=2340 m/s, are c_{2T}=912 m/s are used. As for the acoustic impedances, Z₁=1485600 kg/m²s, Z₃=1485600 kg/m²s, Z_{2L}=2971800 kg/m²s, and Z_{2T}=1158240 kg/m²s are used.

FIG. 5A illustrates a reception signal obtained by a transducer located at a position for which the incident angle to the holding cup 120 is 47 degrees. FIG. 5B is a corrected reception signal obtained by using Equation (14). FIG. 5C illustrates a comparative example and illustrates a reception signal obtained by the same transducer used as that in FIG. 5A based on a signal from the same sound source in a state the holding cup 120 is removed. The horizontal axis of FIGS. 5A to 5C represents the sampling number, whereas the vertical axis of FIGS. 5A to 5C represents the intensity of the signal. The waveform illustrated in FIG. 5A is distorted, compared to the waveform illustrated in FIG. 5C; however, the corrected reception signal illustrated in FIG. 5B has a waveform closer to the waveform illustrated in FIG. 5C, which implies that the waveform is corrected.

In addition, FIGS. 5A and 5B also indicate that the amplitude of the signal is also increased as a result of conversion from the waveform illustrated FIG. 5A to the waveform illustrated FIG. 5B. This indicates that processing based on Equation (14) effectively compensates for a loss (loss due to reflection) that occurs when the acoustic wave passes through the holding cup 120.

Compensating for a loss that occurs when the acoustic wave passes through the holding cup 120 usually increases the quality of the resultant image. However, if the intensity of the signal is weak and the signal-to-noise (SN) ratio is low, the compensation may relatively enhance noise and consequently decrease the quality of the image. In such a case, the deconvolution filter may be changed so that the waveform is corrected by phase shifting but the amplitude is not corrected. Specifically, the convolution filter D(ω) may be normalized so that

$${\sum }_{ω}^{ } {\vert D ( ω ) \vert}^{2} \qquad ( 15 )$$

gives a constant value.

In addition to the above method, another method of using the complex conjugate of the transmittance filter f(ω) as the deconvolution filter D(ω) may also be employed. This method also effectively corrects the waveform by shifting the phase without correcting the amplitude.

The computer 150 may evaluate the SN ratio of the signal data and may perform deconvolution in which correction of the waveform (phase correction) is performed without correction of the amplitude if the SN ratio of the signal data is equal to or smaller than a threshold. In addition, the computer 150 may perform deconvolution denoted by Equation (14) including amplitude correction as well as phase correction if the SN ratio of the signal data is larger than the threshold.

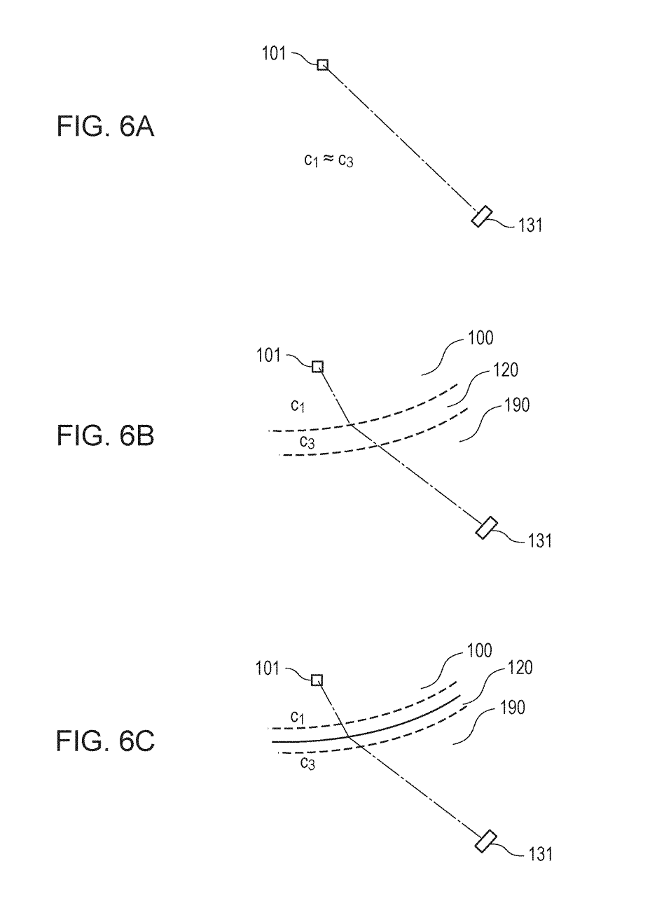

In the case where the sound velocity can be considered to be constant before and after the acoustic wave is incident to the holding cup 120, the corrected reception signal S(t) can be considered to be a signal obtained by the transducer 131 based on an acoustic wave that has been produced at the position corresponding to the specified voxel 101 and has propagated through a space without any boundaries as illustrated in FIG. 6A. In the case where the difference in sound velocity before and after the acoustic wave is incident to the holding cup 120 is taken into account, the corrected reception signal S(t) can be considered to be a reception signal based on an acoustic wave that has propagated as illustrated in FIG. 6B. That is, the corrected reception signal S(t) can be considered to be a signal obtained by the transducer 131 based on an acoustic wave that has been produced at the position corresponding to the specified voxel 101 and has propagated while refracting once at the first surface (surface closer to the subject 100) of the holding cup 120. In FIG. 6B, the holding cup 120 is denoted by dash lines.

When the thickness of the holding cup 120 is sufficiently small, an error is small even if the transmittance filter is obtained by setting a surface of the holding cup 120 as a boundary of the sound velocity as in the first exemplary embodiment. Since calculation is easy in this case, an amount of calculation is small. On the other hand, when the thickness of the holding cup 120 is large, a deviation of the calculated propagation path from the actual propagation path increases. Accordingly, the computer 150 may obtain the transmittance filter by setting a point inside the holding cup 120 as the boundary of the sound velocity as illustrated in FIG. 6C if the thickness of the holding cup 120 is larger than a threshold. The error can be decreased by setting the center of the holding cup 120 in thickness as the boundary of the sound velocity. In FIG. 6C, the holding cup 120 is denoted by dash lines, and the interface of the sound velocity is represented by a solid line. The computer 150 may determine which surface is to be set as the boundary of the sound source on the basis of the thickness of the holding cup 120. For example, the computer 150 may perform calculation by setting a point inside the holding cup 120 as the boundary of the sound velocity if the thickness of the holding cup 120 is larger than 1 mm. On the other hand, the computer 150 may perform calculation by setting the surface of the holding cup 120 as the boundary of the sound velocity if the thickness of the holding cup 120 is equal to or less than 1 mm. When calculation is performed by setting a point (e.g., the center) inside the holding cup 120 as the boundary of the sound velocity as illustrated in FIG. 6C, the “exp” term at the latter part of Equation (11) is to be calculated on the assumption that the subject 100 is present in a half region of the holding cup 120. In addition, the calculation is to be performed on the assumption that the acoustic matching material 190 is present in the other half region of the holding cup 120.

Note that this step is performed for all the transducers 131. Processing of this step may be performed sequentially for the respective transducers 131 in terms of time, or parallel processing may be performed for the plurality of transducers 131 simultaneously.

### S170: Obtaining Subject Information by Using Corrected Signal Data

The computer 150 serving as an image reconstructing unit obtains subject information for the specified voxel 101 by performing reconstruction processing on the corrected reception signal S(t) obtained in S160. As the reconstruction algorithm, a universal back-projection (UBP) algorithm is used. As a result of correction of the reception signal described in S160, the distortion of the waveform caused when the acoustic wave passes through the holding cup 120 is corrected. Accordingly, in this step, reconstruction processing can be performed on the assumption that the signal is obtained in the system illustrated in FIG. 6A, 6B, or 6C. Note that any reconstruction processing may be used as long as subject information is successfully obtained from the corrected signal data.

In the case where the sound velocity is considered to be constant before and after the acoustic wave is incident to the holding cup 120 as illustrated in FIG. 6A, a value obtained by dividing the distance between the specified voxel 101 and the transducer 131 by the longitudinal-wave sound velocity in the subject 100 or the acoustic matching material 190 corresponds to traveling time. Information (amplitude) equivalent to the traveling time in the corrected reception signal S(t) is information from which the subject information of the specified voxel 101 is derived.

In the case where the difference between the sound velocities before and after the acoustic wave is incident to the holding cup 120 is taken into account, the traveling time is determined in the following manner. Specifically, in FIG. 6B, the sum of a value obtained by dividing the distance from the specified voxel 101 to the boundary at which the acoustic wave refracts by the sound velocity in the subject 100 and a value obtained by dividing the distance from the refraction boundary to the transducer 131 by the sound velocity in the acoustic matching material 190 corresponds to traveling time. Information (amplitude) equivalent to the traveling time in the corrected reception signal S(t) is information from which the subject information of the specified voxel 101 is derived. The same applies to the case illustrated in FIG. 6C.

The computer 150 successfully obtains a two-dimensional or three-dimensional spatial distribution of subject information by performing the processing of S140 to S170 for all the voxels for which the subject information is to be obtained.

The computer 150 may obtain a two-dimensional or three-dimensional initial sound pressure distribution as the subject information by performing the processing described above. In addition, the computer 150 may obtain a light fluence distribution in the subject 100 of the light with which the subject 100 is irradiated and may obtain an optical absorption coefficient distribution by using the initial sound pressure distribution and the light fluence distribution. In addition, the computer 150 may obtain a concentration distribution, such as an oxygen saturation distribution, by using the optical absorption coefficient distribution. For example, a concentration distribution can be obtained by using an optical absorption coefficient distribution for light of a plurality of wavelengths. The computer 150 outputs, to the display unit 160, the subject information such as the initial sound pressure distribution, the optical absorption coefficient distribution, or the concentration distribution, obtained in this step.

### S180: Displaying Subject Information

The computer 150 causes the display unit 160 to display the subject information of a region to be imaged, by using the subject information obtained in S170. The display unit 160 is capable of displaying the subject information, such as the initial sound pressure distribution, the optical absorption coefficient distribution, or the concentration distribution (oxygen saturation distribution). Since the subject information displayed on the display unit 160 is information obtained by reducing the distortion of the waveform caused when the acoustic wave passes through the holding cup 120, the displayed information is suitably used by an operator, such as a doctor, to make a diagnosis or the like.

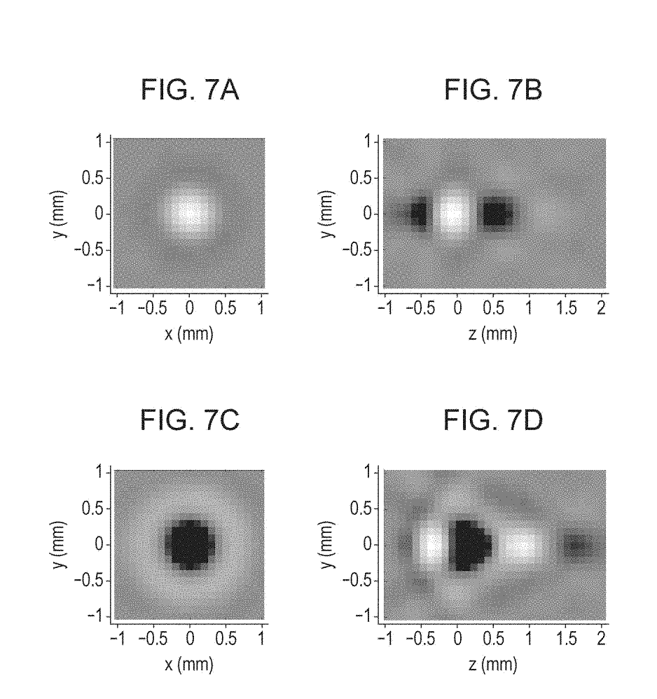

FIGS. 7A to 7D illustrate an example case where photoacoustic image information (three-dimensional subject information) is obtained by using reception signals actually measured. In this example case, a rubber ball having a diameter of 0.3 mm, which serves as a sound source (optical absorber), is placed adjacent to the center of the spherical surface on which the transducers 131 are disposed. The space around the sound source is filled with water.

FIGS. 7A and 7B illustrate photoacoustic image information obtained when the corrected reception signals are used. Referring to FIG. 1, a direction from the light irradiation unit 110 toward the subject 100 (the upward direction in FIG. 1) is set to be the +Z direction, and a plane perpendicular to the +Z direction is set to be an X-Y plane. FIG. 7A illustrates a cross-section on the X-Y plane, whereas FIG. 7B illustrates a cross-section on the Y-Z plane. FIGS. 7C and 7D illustrate photoacoustic image information obtained by using the uncorrected reception signals. FIG. 7C illustrates a cross-section on the X-Y plane, whereas FIG. 7D illustrates a cross-section on the Y-Z plane.

FIGS. 7A to 7D indicate that a point image is successfully reconstructed when the corrected reception signals are used. In contrast, when the uncorrected reception signals are used, the point is separated into two points along the Z direction because of the distortion of the waveform caused when the acoustic wave passes through the holding cup 120.

As described above, the photoacoustic apparatus according to the first exemplary embodiment can obtain highly precise subject information by correcting a distortion of the waveform (in amplitude or phase) caused when an acoustic wave passes through a holding cup.

Although the illustration is omitted, the support 132 may be coupled to a stage, serving as a moving unit, so as to be movable. In this case, subject information is successfully obtained for a subject of a large volume.

### Second Exemplary Embodiment

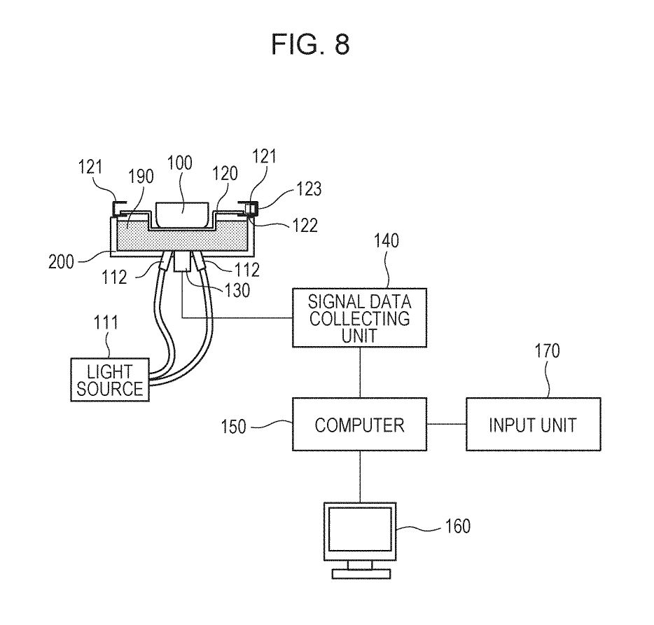

FIG. 8 is a schematic diagram illustrating a photoacoustic apparatus according to a second exemplary embodiment. Note that the same or substantially the same components are denoted by the same reference sign as a rule, and a description thereof is omitted. In the first exemplary embodiment, since a subject having a shape of a breast is assumed as a target of measurement, the holding cup 120 has a hemispherical shape. In the second exemplary embodiment, a subject having a relatively flat shape, such as a hand or a foot, is assumed as the target of measurement, and the holding cup 120 having a flat holding surface is used.

The probe 130 is a receiving unit including transducers arranged in a two-dimensional array. A container 200 stores water, which is the acoustic matching material 190.

A holding plate 120 serving as a holding member is composed of a polyethylene terephthalate resin having a thickness of 0.5 mm. A surface of the holding plate 120 to be in contact with the subject 100 is flat. A space between the subject 100 and the holding plate 120 is filled with ultrasound gel (not illustrated in FIG. 8) for acoustic matching. The holding plate 120 and the probe 130 are configured to have a gap of 30 mm therebetween.

In the second exemplary embodiment, the probe 130 including 10×20 transducers arranged in a two-dimensional array is used. Each of the transducers includes a piezoelectric element having an element size of 2 mm per side and a center detection frequency of 1 MHz.

In the second exemplary embodiment, the signal data collecting unit 140 sets a signal corresponding to a timing at which the subject 100 is irradiated with pulsed light as the 0-th sample and obtains signals of 2048 samples from the 0-th sample to the 2047-th sample.

The inventor has experimentally found that a transmittance filter for which multiple reflections are taken into account is desirably used in the case where the holding surface of the holding plate 120 is flat as in the second exemplary embodiment. That is, in the second exemplary embodiment, a distortion of a transmitted wave occurs because of multiple reflections due to a first surface (surface closer to the subject 100) and a second surface (surface closer to the probe 130) of the holding plate 120 when the acoustic wave passes through the holding plate 120.

Let c₁ denote the (longitudinal-wave) sound velocity in the subject 100, c_{2L} and c_{2T} respectively denote the longitudinal-wave and transversal-wave sound velocities in the holding plate 120, c₃ denote the (longitudinal-wave) sound velocity in the acoustic matching element 190, θ₁ denote the incident angle, and T denote the thickness of the holding plate 120. In addition, let Z₁ denote the acoustic impedance of the subject 100, Z_{2L} and Z_{2T} respectively denote the acoustic impedances of the holding plate 120 for a longitudinal wave and a transverse wave, and Z₃ denote the acoustic impedance of the acoustic matching material 190. The incident angle θ₁ is calculated by the computer 150.

Let θ_{2L} and θ_{2T} respectively denote propagation angles of the longitudinal wave and the transverse wave in the holding plate 120 and θ₃ denote the propagation angle in the acoustic matching material 190. Relationships among these parameters are as described by using Equations (1) to (4).

A transmittance filter f(ω) for an acoustic wave (longitudinal wave) that is incident on the first surface of the holding plate 120 from the side closer to the subject 100 at the incident angle θ₁, passes through the holding plate 120, and propagates toward the acoustic matching material 190 as a longitudinal wave is determined in the following manner. Specifically, the transmittance filter f(ω) can be derived by solving equations by taking into account the continuity of the wave at the first surface and the second surface of the holding plate 120 while assuming a longitudinal wave and a transverse wave that propagate through the holding plate 120 in a certain direction and a longitudinal wave and a transverse wave that propagate in the opposite direction.

When F, G, N, M are defined as represented by Equations (16) to (19), the transmittance filter f(ω) for which multiple reflections are taken into account can be represented by Equation (20).

$$\begin{matrix}F = \frac{{Z}_{2 L} {cos}^{2} 2 {θ}_{2 T}}{cos {θ}_{2 L} sin ( \frac{ω}{{c}_{2 L}} T cos {θ}_{2 L} )} & ( 16 ) \\ G = \frac{{Z}_{2 T} {sin}^{2} 2 {θ}_{2 T}}{cos {θ}_{2 T} sin ( \frac{ω}{{c}_{2 L}} T cos {θ}_{2 T} )} & ( 17 ) \\ N = F + G & ( 18 ) \\ M = F cos ( \frac{ω}{{c}_{2 L}} T cos {θ}_{2 L} ) + G cos ( \frac{ω}{{c}_{2 T}} T cos {θ}_{2 T} ) & ( 19 ) \\ f ( ω ) = \frac{i2 \frac{N cos {θ}_{1}}{{Z}_{3}}}{\frac{cos {θ}_{1} cos {θ}_{3}}{{Z}_{1} {Z}_{3}} ( {M}^{2} - {N}^{2} ) - 1 + iM ( \frac{cos {θ}_{1}}{{Z}_{1}} + \frac{cos {θ}_{3}}{{Z}_{3}} )} exp ( i \frac{ω T cos {θ}_{3}}{{c}_{3}} ) & ( 20 )\end{matrix}$$

The terms F, G, N, and M of Equations (16) to (19) are functions of the angular frequency ω that are introduced to simplify Equation (20). In addition, the “exp” term at the latter part of Equation (20) is provided to correct an amount of phase shift that occurs when the acoustic matching material 190 is present in a region of the holding plate 120. With the presence of this “exp” term, a reception signal that is equivalent to a signal obtained in the case where the region of the holding plate 120 is replaced with the acoustic matching material 190 is obtained when deconvolution processing described in S160 is performed.

Also in the second exemplary embodiment, a high-definition photoacoustic image (spatial distribution of subject information) can be obtained by correcting the distortion of the waveform (in amplitude or phase) caused when an acoustic wave passes through the holding member.

The transmittance filter (equivalent to Equation (11)) used in the first exemplary embodiment is a filter derived on the assumption that there is no multiple reflections in the holding means (first medium). On the other hand, the transmittance filter (equivalent to Equation (20)) used in the second exemplary embodiment is a filter derived on the assumption that multiple reflections ideally occur in the holding member. Incomplete multiple reflections may be desirably taken into account in some cases depending on the shape of the holding member. In such a case, an average of Equations (11) and (20) may be used as the transmittance filter, or a weighted average may be used as a frequency filter. The weight may be adjusted so that the photoacoustic image has the best image quality (e.g., contrast). Note that the user may switch, by using the input unit 170, between a filter for which multiple reflections are taken into account and a filter for which multiple reflections are not taken into account.

### Third Exemplary Embodiment

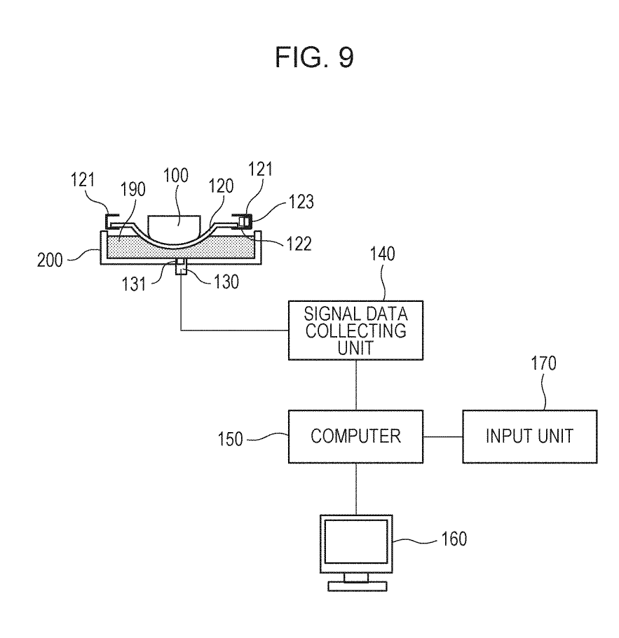

FIG. 9 is a schematic diagram of an ultrasound echo apparatus, which serves as a subject information obtaining apparatus according to a third exemplary embodiment. In the third exemplary embodiment, the probe 130 transmits an ultrasonic wave toward the subject 100 and receives an ultrasonic wave (echo) reflected inside the subject 100. That is, in the third exemplary embodiment, the probe 130 functions as a transmitting-receiving unit that transmits and receives an ultrasonic wave.

The holding cup 120 is composed of a polyethylene terephthalate resin having a thickness of 0.5 mm. The portion of the holding cup 120 to be in contact with the subject 100 is a hemispherical surface having a radius of 230 mm. A space between the subject 100 and the holding cup 120 is filled with ultrasound gel (not illustrated) for acoustic matching.

In the third exemplary embodiment, the probe 130 includes the transducer 131 constituted by a piezoelectric element having a focal distance of 30 mm and a center detection frequency of 6 MHz.

The computer 150 serving as a transmission control unit sends a pulsed signal to the transducer 131 to cause the transducer 131 to transmit an ultrasonic wave having a desired pulse shape. The transmission control unit is typically called a pulser. The signal data collecting unit 140 samples an electric signal obtained by the transducer 131 at a sampling rate of 50 MHz. The signal data collecting unit 140 sets a signal corresponding to a timing at which the transducer 131 transmits a pulsed ultrasonic wave toward the subject 101 as the 0-th sample and stores signals of 2048 samples from the 100-th sample to the 2147-th sample.

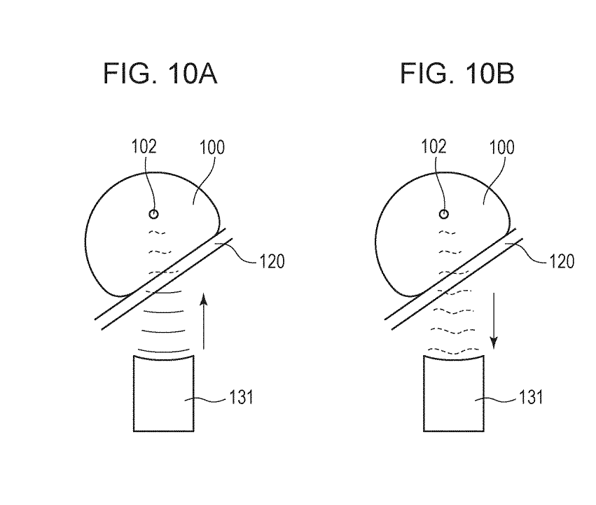

FIG. 10A illustrates a state where an ultrasonic wave is transmitted from the transducer 131 toward the subject 100. The subject 100 includes a reflecting object 102 therein. As described above, a distortion (change in amplitude and phase), such as a change in waveform or a shift in time, occurs in the transmitted ultrasonic wave when the ultrasonic wave passes through the holding cup 120. Part of such a distorted ultrasonic wave hits and is reflected by the reflecting object 102 existing in the subject 100.

FIG. 10B illustrates a state where the distorted ultrasonic wave reflected by the reflecting object 102 reaches and is received by the transducer 131. As in FIG. 10A, a distortion, such as a distortion in waveform or a shift in time, occurs in the reflected ultrasonic wave when the reflected ultrasonic wave passes through the holding cup 120. The ultrasonic wave further distorted is received by the transducer 131. When an ultrasound echo image is obtained from a reception signal obtained in this manner, a resulting image contains the distortion.

The third exemplary embodiment provides an ultrasound echo apparatus capable of correcting such a distortion in a reception signal. Examples of subject information obtained in the third exemplary embodiment include B-mode image data, Doppler-mode image data (blood flow rate distribution), and elastography image data (distortion distribution).

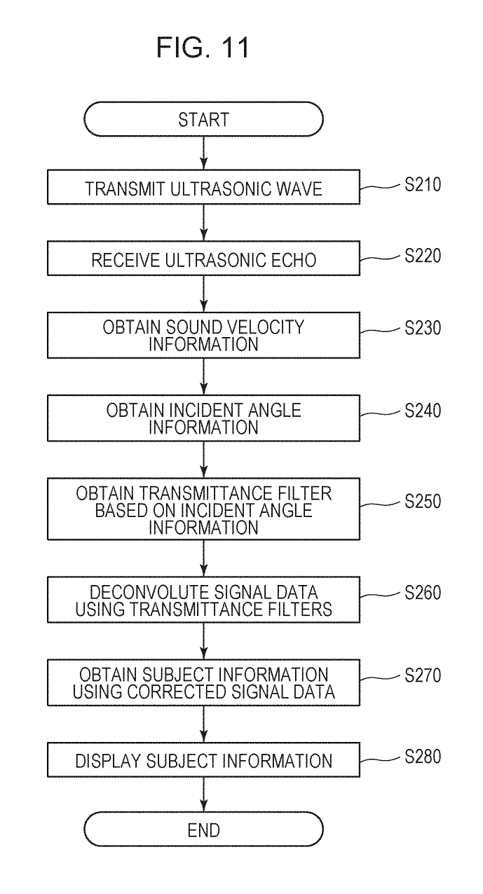

The third exemplary embodiment will be described below with reference to a flowchart for obtaining an ultrasound echo image according the third exemplary embodiment illustrated in FIG. 11.

### S210: Transmitting Ultrasonic Wave

The computer 150 serving as the transmission control unit sends a pulsed signal to the transducer 131 and causes the transducer 131 to transmit an ultrasonic wave having a desired pulse shape. Note that the probe 130 serving as a transmitting unit is disposed such that the subject 100 is irradiated with the transmitted ultrasonic wave after the ultrasonic wave passes through the holding cup 120.

### S220: Receiving Reflected Wave (Echo) of Ultrasonic Wave

The probe 130 receives an ultrasonic echo that occurs as a result of the ultrasonic wave transmitted in S210 being reflected by the reflecting object 102 and outputs an electric signal. The signal data collecting unit 140 converts the electric signal, which is output as an analog signal from the probe 130, into a digital signal and outputs the digital signal to the computer 150.

### S230: Obtaining Sound Velocity Information

The computer 150 serving as the sound velocity obtaining unit obtains a longitudinal-wave sound velocity c₁ in the subject 100, a longitudinal-wave sound velocity c_{2L} in the holding cup 120, a transverse-wave sound velocity c_{2T} in the holding cup 120, and a longitudinal-wave sound velocity c₃ in the acoustic matching material 190 by using a method similar to that used in S130.

### S240: Obtaining Incident Angle Information

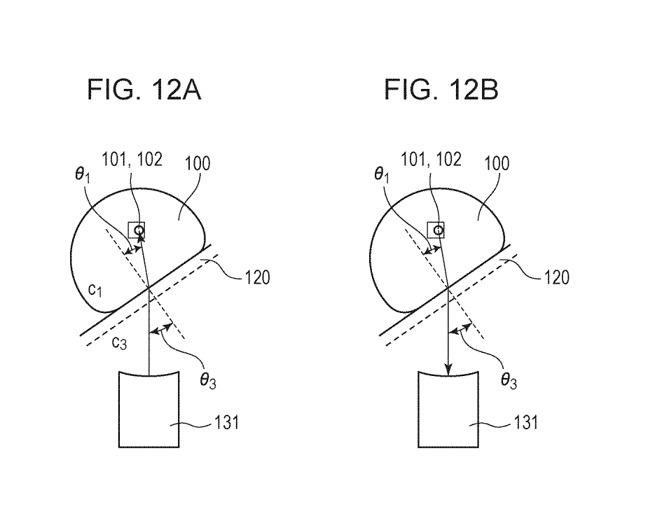

The computer 150 serving as the angle obtaining unit obtains incident angle information regarding an angle at which an ultrasonic echo that is to reach the transducer 131 is incident to the holding cup 120 when the holding cup 120 and the transducer 131 are disposed as illustrated in FIGS. 12A and 12B. In the third exemplary embodiment, an example will be described in which the computer 150 obtains incident angle information regarding an angle at which an ultrasonic echo produced at a position corresponding to the voxel 101 to be imaged later is incident to the holding cup 120. In the third exemplary embodiment, the position of the reflecting object 102 is described as the position corresponding to the voxel 101 (position of interest).

In the third exemplary embodiment, since the transducer 131 is of a focus type, a spherical wave is actually transmitted; however, it is assumed that an ultrasonic wave is transmitted from the center of the transducer 131 in a direction of the principal axis thereof for calculation. FIGS. 12A and 12B illustrate this state. FIG. 12A illustrates the state when an ultrasonic wave is transmitted, whereas FIG. 12B illustrates the state when an ultrasonic echo produced at the position of interest is received. This assumption does not substantially cause any issue unless the transducer 131 has an extremely small focal distance.

The computer 150 obtains an incident angle θ₃ to the first surface (surface closer to the subject 100) of the holding cup 120 that satisfies Snell's law represented by Equation (1) in the transmission case illustrated in FIG. 12A. Since the propagation direction of the ultrasonic wave in FIG. 12A is opposite to the propagation direction of the acoustic wave in FIG. 4C, the incident angle is θ₃ and the propagation angle is θ₁.

The computer 150 also obtains the incident angle θ₁ to the first surface of the holding cup 120 that satisfies Snell's law represented by Equation (1) in the reception case illustrated in FIG. 12B. Since the propagation direction of the ultrasonic echo in FIG. 12B is the same as the propagation direction of the acoustic wave in FIG. 4C, the incident angle is θ₁.

### S250: Obtaining Transmittance Filter by Using Incident Angle Information

The computer 150 serving as the transmittance filter obtaining unit obtains transmittance filters on the basis of the pieces of incident angle information obtained in S240 at the time of transmission and reception.

First, the computer 150 obtains a transmittance filter for the transmission on the basis of the incident angle θ₃ obtained for the transmission. Let c₁ denote the (longitudinal-wave) sound velocity in the subject 100, c_{2L} and C_{2T} respectively denote the longitudinal-wave and transverse-wave sound velocities in the holding cup 120, c₃ denote the (longitudinal-wave) sound velocity in the acoustic matching material 190, and T denote the thickness of the holding cup 120. In addition, let Z₁ denotes the acoustic impedance of the subject 100, Z_{2L} and Z_{2T} respectively denote the acoustic impedances of the holding cup 120 for a longitudinal wave and a transverse wave, and Z₃ denote the acoustic impedance of the acoustic matching material 190. As in the first exemplary embodiment, subscripts 1, 2, and 3 respectively correspond to the subject 100, the holding cup 120, and the acoustic matching material 190.

Let θ_{2L} and θ_{2T} respectively denote the propagation angles of a longitudinal wave and a transverse wave in the holding cup 120. These parameters have relationships described by using Equations (1) to (4).

When multiple reflections are not taken into account, a transmittance filter f₁(ω) of the holding cup 120 for the transmission direction with respect to the angular frequency ω can be represented by Equation (25) by using Equations (21) to (24). In Equation (25), φ_{2L} and φ_{2T} are the same as those of Equations (7) and (8).

$$\begin{matrix}{t}_{32 L} = \frac{2 \frac{{Z}_{3}}{cos {θ}_{2 L}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 21 ) \\ {t}_{32 T} = \frac{- 2 \frac{{Z}_{3}}{cos {θ}_{2 T}} sin 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 22 ) \\ {t}_{21 L} = \frac{2 \frac{{Z}_{2 L}}{cos {θ}_{1}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 23 ) \\ {t}_{21 T} = \frac{- 2 \frac{{Z}_{2 T}}{cos {θ}_{1}} sin 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 24 ) \\ {f}_{1} ( ω ) = ( {t}_{32 L} {φ}_{2 L} ( ω ) {t}_{21 L} + {t}_{32 T} {φ}_{2 T} ( ω ) {t}_{21 T} ) exp ( i \frac{ω T cos {θ}_{3}}{{c}_{3}} ) & ( 25 )\end{matrix}$$

The “exp” term at the latter part of Equation (25) is provided to correct the amount of phase shift that occurs when the acoustic matching material 190 is present in a region of the holding cup 120.

When multiple reflections are taken into account, the transmittance filter f₁(ω) of the holding cup 120 for the transmission direction with respect to the angular frequency ω can be represented by Equation (26). In Equation (26), N and M are the same as those of Equations (18) and (19).

$${f}_{1} ( ω ) = \frac{i 2 \frac{N cos {θ}_{3}}{{Z}_{1}}}{\frac{cos {θ}_{1} cos {θ}_{3}}{{Z}_{1} {Z}_{3}} ( {M}^{2} - {N}^{2} ) - 1 + iM ( \frac{cos {θ}_{1}}{{Z}_{1}} + \frac{cos {θ}_{3}}{{Z}_{3}} )} exp ( i \frac{ω T cos {θ}_{3}}{{c}_{3}} ) \qquad ( 26 )$$

The computer 150 then obtains a transmittance filter for the reception by using the incident angle information for the reception. The propagation direction of the acoustic wave at the time reception is the same as that of the first or second exemplary embodiment. Accordingly, when multiple reflections are not taken into account, a transmittance filter f₂(ω) of the holding cup 120 for the reception direction with respect to the angular frequency ω is the same as the transmittance filter f(ω) represented by Equation (11). When multiple reflections are taken into account, the transmittance filter f₂(ω) of the holding cup 120 for the reception direction with respect to the angular frequency ω is the same as the transmittance filter f(ω) represented by Equation (20).

In deconvolution processing performed in S260 (described later), processing is performed on a frequency-domain reception signal obtained by performing discrete Fourier transform on the reception signal. In the third exemplary embodiment, discrete Fourier transform on the reception signal yields 2048 pieces of frequency information from −9990234.375 Hz to 10000000 Hz at an interval of 9765.625 Hz. Accordingly, the transmittance filter is calculated for the 2048 angular frequencies needed in the processing in S260.

The transmittance filters are obtained on the assumption that the boundary of sound velocity is the same at the time of transmission and at the time of reception in the third exemplary embodiment; however, different boundaries of sound velocity may be set for transmission and reception to obtain transmittance filters. With such a configuration, the accuracy of the transmittance filters is successfully increased. However, if the same boundary of sound velocity is used for transmission and reception, calculation cost can be reduced.

### S260: Performing Deconvolution on Signal Data by Using Transmittance Filters

The computer 150 serving as the correcting unit performs deconvolution on the signal data obtained in S220, by using the transmittance filters f₁(ω) and f₂(ω) respectively for transmission and reception obtained in S250 as response functions. In this way, corrected signal data is obtained. This calculation will be described below.

First, deconvolution filters D₁(ω) and D₂(ω) are respectively determined as represented by Equations (27) and (28).

$$\begin{matrix}{D}_{1} ( ω ) = \frac{conj ( {f}_{1} ( ω ) )}{conj ( {f}_{1} ( ω ) ) \cdot {f}_{1} ( ω ) + C} & ( 27 ) \\ {D}_{2} ( ω ) = \frac{conj ( {f}_{2} ( ω ) )}{conj ( {f}_{2} ( ω ) ) \cdot {f}_{2} ( ω ) + C} & ( 28 )\end{matrix}$$

In Equations (27) and (28), C is a constant and is set empirically so as not to degrade the signal considerably. The user may input the value of the constant C by using the input unit 170.

Let S₀(t) denote the reception signal. Then, the corrected reception signal S(t) can be determined as represented by Equation (29).

S(t)=real(F⁻¹[F[S₀](ω)·D₁(ω)·D₂(ω)]) (29)

The corrected reception signal S(t) thus obtained is substantially equivalent to a reception signal obtained when the region of the holding cup 120 is replaced with the acoustic matching material 190.

### S270: Obtaining Subject Information by Using Corrected Signal Data

The computer 150 serving as the image reconstructing unit obtains subject information for the specified voxel 101 by performing reconstruction processing on the corrected reception signal S(t) obtained in S260. In this step, the computer 150 obtains one-dimensional ultrasound echo image information by performing envelope processing on the corrected reception signal. Note that any reconstruction processing may be used as long as subject information is obtained from the corrected signal data.

In addition, two-dimensional or three-dimensional ultrasound echo image information is obtained by mechanically or electrically moving the probe 130 and repeatedly performing steps of S210 to S270.

The computer 150 outputs, to the display unit 160, the subject information obtained in this step, such as B-mode image data, Doppler-mode image data, or elastography image data.

### S280: Displaying Subject Information

The computer 150 causes the display unit 160 to display the subject information of a region to be imaged, by using the subject information obtained in S270. The display unit 160 is capable of displaying the subject information such as B-mode image data, Doppler-mode image data, or elastography image data. Since the subject information displayed on the display unit 160 is information obtained by reducing the distortion of the waveform caused when the ultrasonic wave passes through the holding cup 120, the displayed information is suitably used by an operator, such as a doctor, to make a diagnosis or the like.

In the third exemplary embodiment, a high-definition ultrasound echo image is successfully obtained by correcting a distortion of a waveform (in amplitude or phase) caused when an ultrasonic wave passes through the holding cup.

### Fourth Exemplary Embodiment

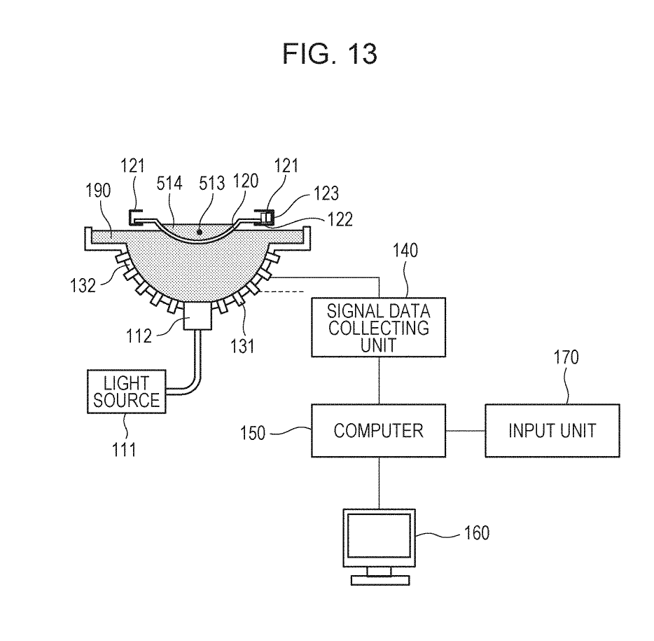

A fourth exemplary embodiment will be described by using a photoacoustic apparatus illustrated in FIG. 13. A component that is the same or substantially the same as an already-described component is denoted by the same reference sign in FIG. 13, and a detailed description thereof is omitted. It is important to accurately grasp the positions of the transducers 131 in order to obtain highly accurate subject information in the photoacoustic apparatus, which is a subject information obtaining apparatus. In the fourth exemplary embodiment, a description is given of a method for obtaining information of the position of each of the transducers 131 by using the corresponding corrected signal obtained in S160 described above.

In the fourth exemplary embodiment, a sound source 513 is placed at a certain position in the holding cup 120. A rubber ball having a diameter of 0.3 mm is used as the sound source 513. The sound source 513 is desirably placed at the spherical center of the support 132 having a spherical surface on which the transducers 131 are disposed. The position of the sound source 513 need not necessarily be the spherical center; however, coordinates of the position of the sound source 513 are desirably known. The space around the sound source 513 is filled with water 514. Any material that absorbs wavelength of a laser beam may be used as the material of the sound source 513. Ideally, the sound source 513 is desirably a spherical body. The sound source 513 need not necessarily be a spherical body and just needs to have a size (the largest value from among the longitudinal width, the horizontal width, and the height) that is shorter than or equal to a wavelength of an acoustic wave having the center frequency of a detection band of the transducers 131 in water. In this case, since the waveform of the acoustic wave produced at the sound source 513 is substantially identical to the waveform of the acoustic wave produced at the spherical body, the sound source 513 can be substantially considered to be substantially spherical. That is, the center frequency of the reception band of the transducers 131 may be set to a center frequency for which the wavelength of the acoustic wave having the center frequency in water is larger than the largest value of the size of the sound source 513.

When the sound source is large, emitted light is absorbed at the surface of the sound source and does not reach inside the sound source in some cases. Accordingly, the sound source can be handled as a hollow optical absorber whose surface alone has an optical absorption effect. An acoustic wave produced at the sound source has a shape closer to an impulse function. For example, the case of using a metal ball, such as an iron ball, corresponds to this case. In this case, when an acoustic wave that is produced at the sound source and propagates in a desired direction and an acoustic wave that propagates in the opposite direction and is reflected on the back surface of the sound source interfere with each other and the resulting wave is detected by the probe, an error occurs in terms of time. Accordingly, the diameter of the sound source is desirably at least 5 times larger than the wavelength of the acoustic wave having the center frequency of the reception band of the transducers in water. The center frequency of the reception band of the transducers 131 may be set to a center frequency for which the wavelength of the acoustic wave having the center frequency in water is ⅕ the largest value of the size of the sound source 513.

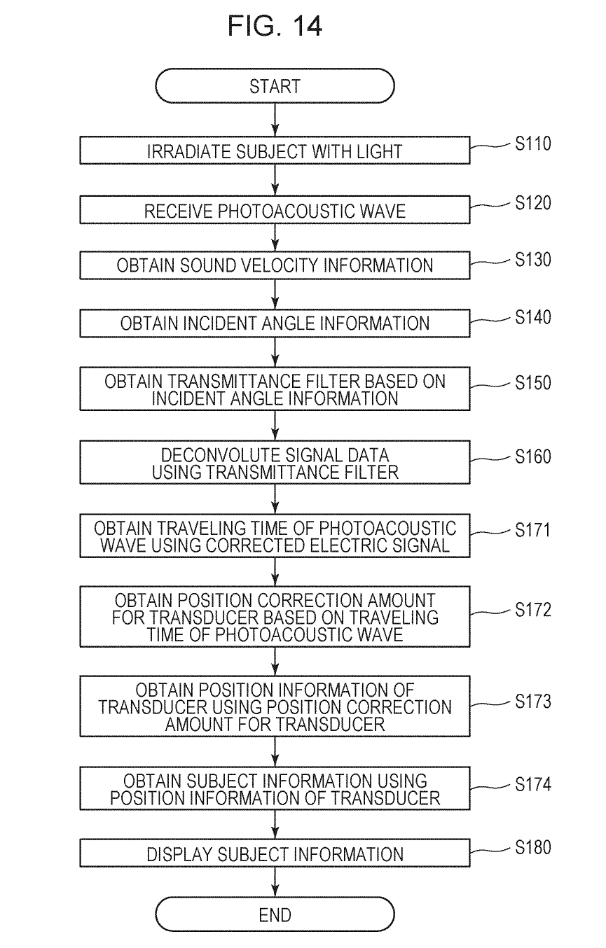

FIG. 14 is a flowchart of obtaining position information of each of the transducers 131 by using an acoustic wave produced at the sound source 513 whose position is known. FIG. 14 illustrates the processing flow after the corrected electric signal is obtained in S160 described above.

### S171: Obtaining Traveling Time of Acoustic Wave by Using Corrected Electric Signal

The computer 150 calculates traveling time taken for the acoustic wave produced at the sound source 513 to reach the transducer 131, by using the corrected electric signal obtained in S160. This calculation will be described below.

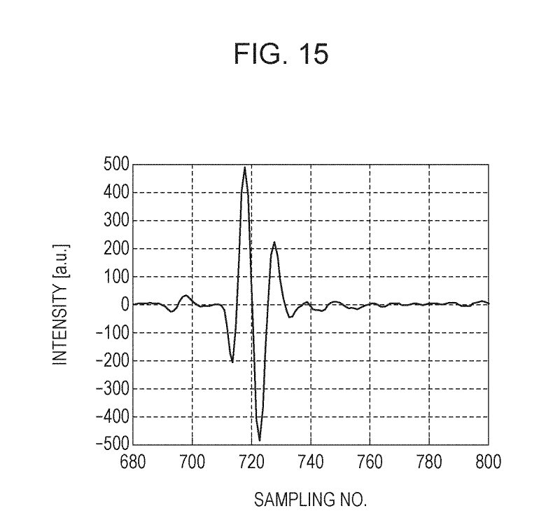

FIG. 15 illustrates an example of the corrected reception signal obtained in S160. The horizontal axis represents the sampling number, whereas the vertical axis represents intensity of the signal. As illustrated in FIG. 15, the corrected reception signal has a point-symmetrical shape with respect to the center position between the largest point and the smallest point of the signal intensity. Accordingly, the sampling number for which the signal intensity is equal to zero between the largest point and the smallest point is set as a reference point of the traveling time. The sampling number is a discrete integer; however, in order to increase the precision, the value of the place after the decimal point is also calculated by performing linear interpolation by using values preceding and following the point at which the graph crosses the X axis. Needless to say, the interpolation method to be used is not limited to this one. Let Nᵢ denote the derived sampling number, where i denotes the element number of the transducer 131.

As described in the first exemplary embodiment, the signal data collecting unit 140 obtains signals of 2048 samples from the 1000-th sample to the 3047-th sample by setting the signal corresponding to the timing at which the sound source 513 is irradiated with the pulsed light to be the 0-th sample. Let fₛₐₘₚ denote the sampling frequency. Then, the traveling time t₁ of the acoustic wave is represented by Equation (30).

tᵢ=(Nᵢ+1000)/fₛₐₘₚ (30)

### S172: Obtaining Position Correction Amount for Transducer Based on Traveling Time of Acoustic Wave

The computer 150 serving as a shift amount estimating unit calculates a position correction amount for the transducer 131 on the basis of the traveling time tᵢ of the acoustic wave obtained in S171. That is, the computer 150 calculates, as the position correction amount, a shift amount of the position of the transducer 131 from the designed position (predetermined position) of the transducer 131. This calculation will be described below.

Let (X₀, Y₀, Z₀) denote the position of the sound source 513 and (xᵢ, yᵢ, zᵢ) denote the designed position of the transducer 131-i. Then, a position correction amount ΔRᵢ can be represented by Equation (31).

ΔRᵢ=tᵢc−\sqrt{(xᵢ−X₀)²+(yᵢ−Y₀)²+(zᵢ−Z₀)²} (31)

In Equation (31), c denotes the sound velocity in water. The position correction amount ΔRᵢ indicates a difference between the designed distance and the actual distance. The position correction amount ΔRᵢ is stored in the storage unit of the computer 150 and is used when image reconstruction (described later) is performed.

### S173: Obtaining Subject Information by Using Position Correction Amount for Transducer

The computer 150 obtains position information of the transducer 131 by using the position correction amount obtained in S172 for the transducer 131. The position correction amount ΔRᵢ is used in the following manner when an image is generated through image reconstruction.

Suppose that R denotes the designed radius (127 mm in the first exemplary embodiment) of the support 132 having a hemispherical surface on which the transducers 131 are disposed and that the designed position of the transducer 131-i is represented by Expression (32) in the spherical coordinate system when the center of the hemispherical surface is set as the origin.

(R sin θᵢ cos φᵢ,R sin θᵢ sin φᵢ,R cos θᵢ) (32)

In this case, the computer 150 determines the actual position of the transducer 131-i as represented by Expression (33).

((R+ΔR)sin θᵢ cos φᵢ,(R+ΔR)sin θᵢ sin φᵢ,(R+ΔR)cos θᵢ) (33)

That is, the computer 150 performs correction by assuming the error in distance as a shift in the radial direction. The fourth exemplary embodiment assumes a fabrication process in which holes for installing the transducers 131 are created in the support 132 and the transducers 131 are inserted and fixed to the respective holes. The error correction represented by Expression (33) is for coping with the fact that the radial direction is a direction for which the error is most likely to occur in this fabrication process.

The above-described method is the method for obtaining the position information of each of the transducers 131 when the position of the sound source 513 is known. According to the method described above, the position information of each transducer is successfully obtained accurately by using a signal obtained by correcting a distortion of the acoustic wave that occurs when the acoustic wave passes through the holding cup.

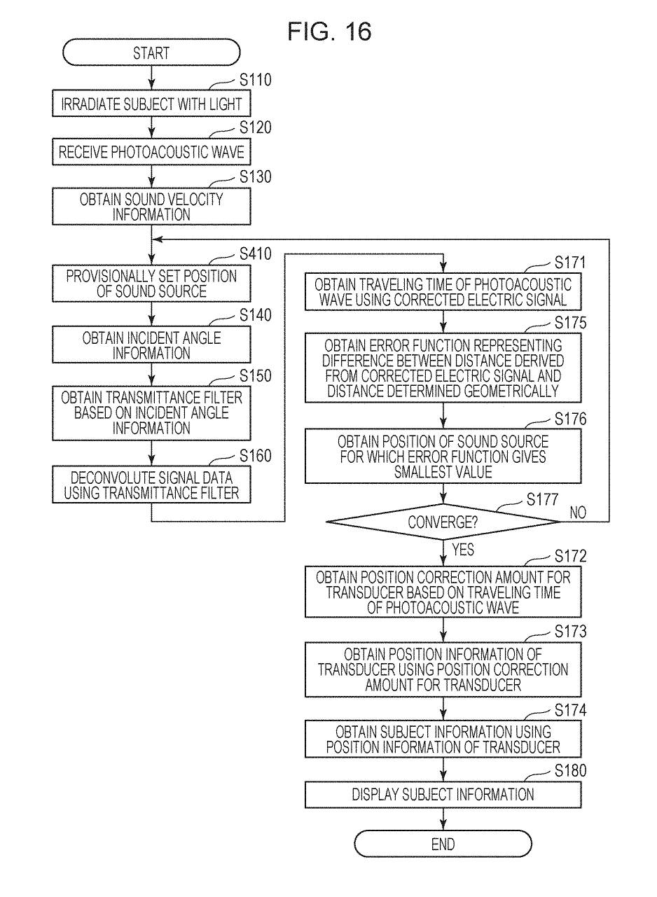

According to the method described above, the accuracy of the determined position of the sound source 513 is dependent on the accuracy of the position information of the transducers 131. Accordingly, a description will be given of a method for obtaining the position information of the transducer 131 when the position of the sound source 513 is not accurately grasped. That is, a description will be given of a method for obtaining the position information of the transducer 131 when the position of the sound source 513 is not known, that is, unknown. FIG. 16 illustrates the flow of the method for obtaining the position information of each of the transducers 131 when the position of the sound source 513 is unknown.

### S410: Provisionally Setting Position of Sound Source

The computer 150 provisionally sets the position of the sound source 513. For example, the computer 150 provisionally sets the position of the sound source 513 to be (xₚ, yₚ, zₚ). Processing is performed in S140, S150, S160, and S171 on the assumption that the sound source 513 is disposed at the position (xₚ, yₚ, zₚ) provisionally set in this step.

S175: Obtaining Error Function Representing Difference Between Distance Derived from Corrected Signal and Distance Derived Geometrically

The computer 150 obtains a distance between the sound source 513 and the transducer 131 on the basis of the traveling time obtained in S171. The computer 150 also obtains a distance between the sound source 513 and the transducer 131 on the basis of the position of the sound source 513 provisionally set in S410 and a designed value representing the position of the transducer 131. Then, the computer 150 obtains an error function representing a difference between these distances. Specifically, an Error function E(v, x, y, z) is defined by Equation (34).

$$E ( v , x , y , z ) = {\sum }_{i}^{ } {( {t}_{i} v - \sqrt{{( {x}_{i} - x )}^{2} + {( {y}_{i} - y )}^{2} + {( {z}_{i} - z )}^{2}} )}^{2} \qquad ( 34 )$$

In Equation (34), v is a variable representing the sound velocity in water. The alphabet v is used to distinguishing this parameter from the constant c. In addition, tᵢ denotes the traveling time of the acoustic wave represented by Equation (30), and x, y, and z are variables representing the position of the sound source 513.

S176: Obtaining Position of Sound Source for which Error Function Gives Smallest Value

Values of v, x, y, and z that minimize the error function E represented by Equation (34) are determined. This is done by determining v, x, y, and z that satisfy

$$\frac{dE}{dv} = 0 , \frac{dE}{dx} = 0 , \frac{dE}{dy} = 0 , \frac{dE}{dz} = 0. \qquad ( 35 )$$

The solutions thus obtained are denoted by vₐ, xₐ, yₐ, and zₐ.

### S177: Determining Whether Convergence is Achieved

The computer 150 determines whether the position (xₐ, yₐ, zₐ) of the sound source 513 obtained in S175 is a converged value.

If it is determined that the value has converged, the process proceeds to S172. The solutions for which it is determined that the value has converged are denoted by Vₐ, Xₐ, Yₐ, and Zₐ. In S172, the computer 150 calculates the position correction amount for the transducer 131 on the basis of the traveling time tᵢ of the acoustic wave, which is obtained in S171 when the value has converged. The position correction amount ΔRᵢ can be represented by Equation (36).

ΔRᵢ=tᵢVₐ−\sqrt{(xᵢ−Xₐ)²+(yᵢ−Yₐ)²+(zᵢ−Zₐ)²} (36)

In Equation (36), (Xₐ, Yₐ, Zₐ) denotes the position of the sound source 513 and (xᵢ, yᵢ, zᵢ) denotes the designed position of the transducer 131-i.

If it is determined that the value has not converged, the process returns to S410. In S410 for the second or following loop, the position (xₐ, yₐ, zₐ) of the sound source 513 obtained in S176 is provisionally set as the position of the sound source 513. Then, processing is performed in S140, S150, S160, and S171 on the assumption that the sound source 513 is disposed at the position (xₐ, yₐ, zₐ) provisionally set.

For example, the computer 150 may determine that the value has converged when the distance between the position (xₐ, yₐ, zₐ) of the sound source 513 obtained in S176 and the position (xₚ, yₚ, zₚ) provisionally set in S410 is smaller than or equal to a threshold. In addition, the computer 150 may determine that the value has converged when the distance between the position (xₐ, yₐ, zₐ) of the sound source 513 obtained in S176 and the position (xₐ, yₐ, zₐ) of the sound source 513 obtained in the immediately preceding loop is smaller than or equal to a threshold. In addition, the computer 150 may determine that the value has converged after the computer 150 has performed the steps of S410 to S176 a predetermined number of times. The threshold regarding the distance and the number of times of repetitions may be specified by the user by using the input unit 170.

In the fourth exemplary embodiment, the sound velocity v is set as a variable; however, the sound velocity may be set to be constant instead of using a variable. In addition, an error function represented by Equation (37) in which T is a variable may be defined in order to adjust a temporal shift between light-emission timing (generation timing of the acoustic wave) and the sampling start timing.

$$E ( v , x , y , z ) = {\sum }_{i}^{ } {( ( {t}_{i} + T ) v - \sqrt{{( {x}_{i} - x )}^{2} + {( {y}_{i} - y )}^{2} + {( {z}_{i} - z )}^{2}} )}^{2} \qquad ( 37 )$$

In addition, when a relatively large spherical body is used as the sound source 513, an acoustic wave is not produced at the center of the sound source 513 but is produced at the surface thereof. Accordingly, propagation time for the distance of the radius needs to be taken into account when the position correction amount ΔRᵢ is calculated. Let r₀ denote the radius of the sound source 513 and (X₀, Y₀, Z₀) denote the center position of the sound source 513. Then, the position correction amount ΔRᵢ can be represented by Equation (38).

ΔRᵢ=tᵢc−({square root over (xᵢ−X₀)²+(yᵢ−Y₀)²+(zᵢ−Z₀)²)}−r₀) (38)

The above-described method is the method for obtaining the position information of each of the transducers 131 when the position of the sound source 513 is unknown. According to the fourth exemplary embodiment, the position information of each transducer is successfully obtained accurately by using a signal obtained by correcting a distortion of the acoustic wave that occurs when the acoustic wave passes through the holding cup.

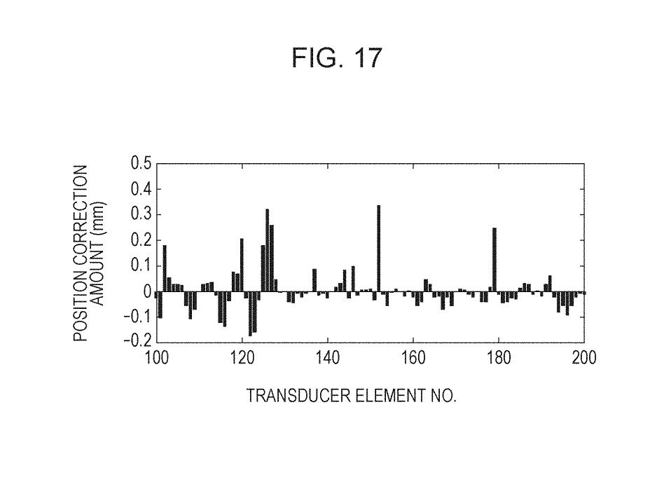

FIG. 17 illustrates an example in which the position correction amount is actually obtained by using the method according to the fourth exemplary embodiment. Specifically, FIG. 17 illustrates the position correction amount calculated by using a rubber ball having a diameter of 0.3 mm as the sound source 513. The horizontal axis represents the element number of the transducer 131 and indicates the 100-th transducer to the 200-th transducer among 512 transducers. The vertical axis represents the position correction amount corresponding to each element number.

In the fourth exemplary embodiment, the case of using a rubber ball having a diameter of 0.3 mm as the sound source 513 has been described; however, the sound source 513 is not limited to the rubber ball. For example, a metal ball, such as an iron ball, may be used the sound source 513.

### Fifth Exemplary Embodiment

It is important to accurately grasp the thickness of the holding cup 120 in order to obtain high-definition subject information in the subject information obtaining apparatuses according to the first to fourth exemplary embodiments. Examples of a method for forming a resin material into a shape suitable for the holding cup 120 include vacuum forming and pressure forming. Both of the methods are methods in which a sheet-like resin material is heated to be softened and then is molded and cooled. Such forming processes may cause a film thickness distribution in the holding cup 120. The film thickness distribution is dependent on various conditions, such as heating temperature and speed of molding. Accordingly, the thickness may become larger at the center than at the periphery in some cases or conversely the thickness may become smaller at the center than at the periphery in some cases. To implement an even film thickness distribution, conditions of the forming method need to be optimized, which can be a factor of increased cost. Even if a holding cup having an uneven film thickness distribution is used, correction of the reception signal described in the first to fourth exemplary embodiments can be performed accurately as long as the film thickness distribution is grasped in advance. However, a special apparatus is needed to measure the thickness of the curved surface, such as the holding cup 120, in a nondestructive manner.

Accordingly, in the fifth exemplary embodiment, a method for obtaining information regarding the thickness of the holding cup 120 in a nondestructive manner by using the photoacoustic apparatus illustrated in FIG. 13 will be described.

First, steps S110 and S120 are performed in the state where the holding cup 120 is placed as illustrated in FIG. 13, and reception signal data is stored in the computer 150. The reception signal data obtained in the state where the holding cup 120 is placed is referred to as first reception signal data.

Then, steps S110 and S120 are performed in the state where the holding cup 120 is removed, and reception signal data obtained in this state is stored in the computer 150. The reception signal data obtained in the state where the holding cup 120 is removed is referred to as second reception signal data. The measurement without the holding cup 120 may be performed first.

Then, the computer 150 provisionally sets data regarding the thickness of the holding cup 120. The data regarding the thickness may be data representing a uniform thickness or data containing a thickness distribution.

Then, the computer 150 obtains a transmittance filter according to Equation (11) or (20) by using the provisionally set data regarding the thickness of the holding cup 120. Then, the computer 150 obtains corrected reception signal data by performing deconvolution on the first reception signal data by using the obtained transmittance filter as in the method described in S160. The corrected reception signal data is referred to as third reception signal data.

Then, the computer 150 obtains traveling time t₂ of the acoustic wave in the case where the holding cup 120 is removed, by using the second reception signal data as in the method described in S171. The computer 150 also obtains traveling time t₃ of the acoustic wave in the case where it is assumed that the holding cup 120 is removed, by using the third reception signal data in the similar manner.

Then, the computer 150 calculates a difference Δt between the traveling time t₂ obtained based on the second reception signal data and the traveling time t₃ obtained based on the third reception signal data. The computer 150 then updates the data regarding the thickness of the holding cup 120 and repeatedly performs the above-described processing until the difference Δt becomes smaller than 0 or a threshold. The computer 150 obtains, as the true value, the data regarding the thickness of the holding cup 120 at the time when the difference Δt becomes smaller than 0 or a threshold. The computer may employ data obtained by performing the update of the data regarding the thickness of the holding cup 120 a predetermined number of times as the true value.

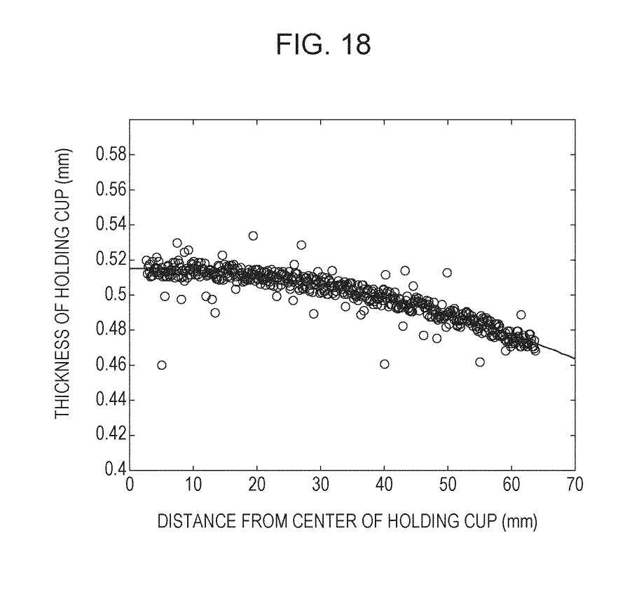

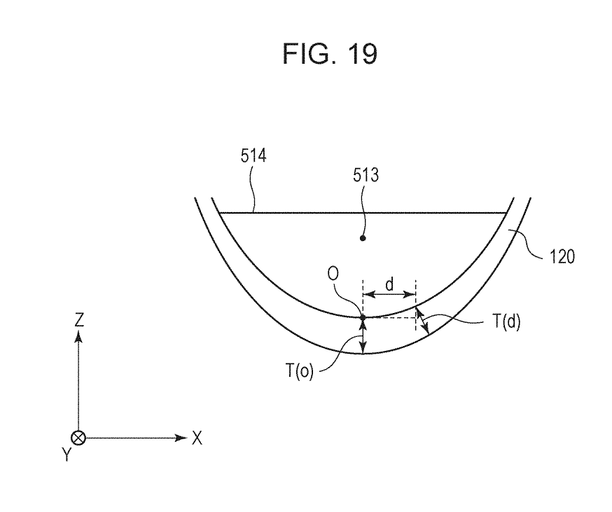

FIG. 18 illustrates a measurement result of the thickness of the holding cup 120 actually obtained by using the method according to the fifth exemplary embodiment. The horizontal axis represents the distance from the center of the holding cup 120, whereas the vertical axis represents the thickness of the holding cup 120. In the fifth exemplary embodiment, a holding cup whose thickness has a distribution depending on the distance from the center is used as the holding cup 120. In FIG. 18, circles represent actually measured points and a solid line is a fitting curve. In this example case, a distance d in an R direction (R=√(X²+Y²)) from the bottom O of the holding cup 120 illustrated in FIG. 19 is set as the distance from the center of the holding cup 120. In addition, thickness of the holding cup 120 in a direction of a normal to the surface of the holding cup 120 is set as a thickness T(d) of the holding cup 120 at the distance d. In the fifth exemplary embodiment, calculation is performed on the assumption that the position of the holding cup 120 is known. That is, in the fifth exemplary embodiment, calculation is performed on the assumption that the position of the surface of the holding cup 120 closer to the sound source 513 is known.

In the fifth exemplary embodiment, the thickness T(d) (mm) is determined as Equation (39), where d is the distance from the center of the holding cup 120.

T(d)=0.511−1.05d²·10⁻⁵ (39)

This information regarding thickness is stored in the storage unit of the computer 150. In the fifth exemplary embodiment, calculation is performed on the assumption that the thickness of the holding cup 120 changes towards the X and Y directions; however, any change in the thickness of the holding cup 120 may be handled.

According to the fifth exemplary embodiment, information regarding the thickness of the holding cup is successfully obtained accurately even if a distortion occurs in an acoustic wave when the acoustic wave passes through the holding cup. In addition, according to the fifth exemplary embodiment, a transmittance filter is successfully obtained accurately even if the thickness of the holding cup is unknown.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-075486 filed Apr. 4, 2016, which is hereby incorporated by reference herein in its entirety.

What is claimed is:

1. A processing system that processes an electric signal obtained as a result of a receiving unit receiving an acoustic wave that has been produced in a subject and has passed through a first medium, the processing system comprising:
a transmittance filter obtaining unit configured to obtain a transmittance filter representing complex transmittances corresponding to a plurality of frequencies in a case where the acoustic wave passes through the first medium; and
a correcting unit configured to correct the electric signal by using the transmittance filter and to obtain a corrected electric signal.

2. The processing system according to claim 1, wherein the correcting unit performs deconvolution on the electric signal by using the transmittance filter and obtains the corrected electric signal.

3. The processing system according to claim 2, further comprising:
an angle obtaining unit configured to obtain incident angle information regarding an incident angle at which the acoustic wave that has been produced at a position of interest in the subject and is to reach the receiving unit is incident to the first medium,
wherein the transmittance filter obtaining unit obtains the transmittance filter on the basis of the incident angle information.

4. The processing system according to claim 3, wherein the transmittance filter obtaining unit calculates the transmittance filter on the basis of the incident angle information.

5. The processing system according to claim 4, further comprising:
a storage unit in which a relationship table or relationship formula is stored, the relationship table or relationship formula representing a relationship between an incident angle and a transmittance filter,
wherein the transmittance filter obtaining unit refers to the relationship table or relationship formula on the basis of the incident angle information and obtains the transmittance filter corresponding to the incident angle indicated by the incident angle information.

6. The processing system according to claim 3, further comprising:
a sound velocity obtaining unit configured to obtain a sound velocity in the subject and a sound velocity in a second medium located between the first medium and the subject,
wherein the angle obtaining unit obtains the incident angle information by using the sound velocity in the subject and the sound velocity in the second medium.

7. The processing system according to claim 6, further comprising:
a storage unit in which the sound velocity in the second medium is stored,
wherein the sound velocity obtaining unit:
reads and obtains the sound velocity in the second medium from the storage unit, and
obtains the sound velocity in the subject by using the electric signal and the sound velocity in the second medium.

8. The processing system according to claim 1, further comprising:
a storage unit in which a relationship table or relationship formula is stored, the relationship table or relationship formula representing a relationship between a transmittance filter and a positional relationship between a position of interest in the subject and a position of the receiving unit; and
a position obtaining unit configured to obtain information regarding the positional relationship between the position of interest and the position of the receiving unit,
wherein the transmittance filter obtaining unit refers to the relationship table or relationship formula on the basis of the information regarding the positional relationship and obtains the transmittance filter corresponding to the positional relationship.

9. The processing system according to claim 8, further comprising:
a position determining unit configured to determine the position of the receiving element,
wherein the position obtaining unit obtains the positional relationship by using the position of the receiving unit determined by the position determining unit.

10. The processing system according to claim 3, further comprising: a subject information obtaining unit configured to obtain subject information corresponding to the position of interest by using the corrected electric signal.

11. The processing system according to claim 8, further comprising:
a position determining unit configured to determine the position of the receiving unit; and
a shift amount estimating unit,
wherein the receiving unit includes at least one receiving element configured to receive an acoustic wave produced in the subject including a certain sound source and to output the electric signal,
wherein the position obtaining unit:
obtains, by using position information regarding the position of the receiving unit determined by the position determining unit, a distance between the at least one receiving element and the certain sound source by assuming that the at least one receiving element is disposed at a predetermined position, and
estimates a distance between the at least one receiving element and the certain sound source on the basis of the corrected electric signal, and
wherein the shift amount estimating unit estimates a shift amount of the position of the at least one receiving element from the predetermined position, on the basis of the distance estimated on the basis of the corrected electric signal and the distance obtained by assuming that the at least one receiving element is disposed at the predetermined position.

12. The processing system according to claim 1, wherein the correcting unit:
evaluates a signal-to-noise ratio of the electric signal,
performs, in response to the signal-to-noise ratio being smaller than a threshold, deconvolution in which phase correction is performed but amplitude correction is not performed on the electric signal on the basis of the transmittance filter and obtains the corrected electric signal, and
performs, in response to the signal-to-noise ratio being larger than the threshold, deconvolution in which both phase correction and amplitude correction are performed on the electric signal on the basis of the transmittance filter and obtains the corrected electric signal.

13. The processing system according to claim 1, wherein the transmittance filter obtaining unit changes the transmittance filter in accordance with information regarding thickness of the first medium.

14. The processing system according to claim 1, further comprising:
a light irradiation unit configured to irradiate the subject with light; and
the receiving unit configured to receive an acoustic wave produced as a result of the subject being irradiated with the light by the light irradiation unit and to output the electric signal.

15. The processing system according to claim 1, further comprising:
a transmitting-receiving unit configured to transmit an ultrasonic wave toward the subject via the first medium and to receive the ultrasonic wave from the subject via the first medium,
wherein the transmitting-receiving unit receives, as the acoustic wave, the ultrasonic wave reflected inside the subject and outputs the electric signal, and
wherein the transmittance filter obtaining unit obtains the transmittance filter in cases where the ultrasonic wave passes through the first medium when being transmitted and when being received.

16. The processing system according to claim 14, wherein a center frequency of a reception band of the receiving unit is a frequency for which a wavelength of an acoustic wave having the center frequency in water is larger than the size of a certain sound source included in the subject.

17. The processing system according to claim 14, wherein a center frequency of a reception band of the receiving unit is a frequency for which a wavelength of an acoustic wave having the center frequency in water is ⅕ the size of a certain sound source included in the subject.

18. The processing system according to claim 14, wherein the first medium is a holding member configured to hold the subject.

19. A signal processing method for processing an electric signal derived from an acoustic wave that has been produced in a subject and has passed through a first medium, the signal processing method comprising:
obtaining a transmittance filter representing complex transmittances corresponding to a plurality of frequencies in a case where the acoustic wave passes through the first medium; and
correcting the electric signal by using the transmittance filter and obtaining a corrected electric signal.

20. A non-transitory storage medium storing a program that causes a computer to perform a signal processing method for processing an electric signal derived from an acoustic wave that has been produced in a subject and has passed through a first medium, the signal processing method comprising:
obtaining a transmittance filter representing complex transmittances corresponding to a plurality of frequencies in a case where the acoustic wave passes through the first medium; and
correcting the electric signal by using the transmittance filter and obtaining a corrected electric signal.
